(19) United States
(12) Patent Application Publication
(10) Pub. No.: US 2004/0126776 A1
(43) Pub. Date: Jul. 1, 2004

(54) Gene library and a method for producing the same

(76) Inventors: Michael Hofer, Heidelberg (DE); Martin Hofmann, Heidelberg (DE); Carmen Kaiser, Dossenheim (DE); Harald Kranz, Leimen (DE); Ralf Lobbert, Heidelberg (DE)

Correspondence Address: McDermott Will & Emery, 600 13th Street NW, Washington, DC, 20005-3096, US

(21) Appl. No.: 10/469,285

(22) PCT Filed: Aug. 28, 2003
(86) PCT No.: PCT/EP02/01849

(30) Foreign Application Priority Data
Feb. 28, 2001 (DE) 201 03 510.3
May 17, 2001 (EP) 01112165.4

(51) Int. Cl. C12Q 1/68; C12P 19/34

(52) U.S. Cl. 435/6; 435/91.2

(57) ABSTRACT

The invention relates to a method for producing a gene library, preferably a cDNA gene library and to a gene library that can be obtained by said method.

The present invention relates to a method for producing a gene library, preferably a cDNA gene library, and to a gene library that can be obtained by said method.

A gene library constitutes a collection of recombinant DNA molecules in the form of e.g. bacteria/plasmid clones, phage lysates, etc. In the ideal case, the respective vector inserts represent the complete genetic information for example of a special organism or tissue, whereat the probability to find a specific gene within such a collection is dependent on the size of the insert, the total genome size and the copy number of the respective sequence element, e.g. the gene, in the genome. cDNA libraries offer the advantage, that they only comprise genes, which are actually expressed and which are free of the intron parts. Moreover, cDNA libraries can be relied upon when investigating the gene expression in specific cells and/or tissues in response to specific factors, e.g. the status of differentiation. However, the problem, that the respective whole gene or the desired parts of the gene are not present within the gene library is frequent to arise in consequence of the primers used for cDNA synthesis from the mRNA template.

In any case, the currently used methods to produce cDNA libraries are laborious, hardly efficient and impaired by a series of further disadvantages, which are discussed in the following. It should be mentioned, that the major difficulty in using the ordinarily produced cDNA libraries for investigating gene expression by means of microarrays is to be found both in un-specific cross-reactions of the spotted cDNA clones—irrespective of being obtained as plasmids or PCR amplification products—when hybridizing with the RNA-samples, and in a poor signal to noise ratio. Both factors may lead to a complete misinterpretation of hybridization results and thus of the whole experiment. Specifically, these problems may be due to various reasons:

a) Conserved domains in the coding sequences of particular members of a gene family lead to a non-specific cross-reaction.

b) Since in general one has to use amplification primers, which correspond to the employed vector in order to produce the PCR-amplified products, normally vector parts are co-amplified. This can lead both to an unspecific reaction and to a significant deterioration of the signal to noise ratio.

c) The commonly employed amplification primers display a relatively low melting temperature (Tm). Accordingly, the amplification reaction has to be accomplished at a rather moderate annealing temperature. The risk of an non-specific binding of the amplification primers and in consequence the risk of a non-specific amplification is thus quite high.

d) The different cDNA-molecules display poly(A)-tails of variable length, which may lead both to an increased background and to a non-specific hybridization reaction.

e) When using the ordinarily produced cDNA libraries for the production of microarrays, the cDNA-inserts are in general present in a non-orientated fashion within the polylinker of a vector system. It is thus impossible to spot only the “antisense”-strand by using just one modified PCR-Primer. This also adds to the significant deterioration in the signal to noise ratio.

f) The extremely variable sizes of the different cDNAs, which are ranging between 500 bp and >5 kb, cause very inhomogeneous yields in the PCR. To allow a representative hybridization experiment however, the cDNA-microarray must be spotted with homogeneous, equimolar amounts of the distinct clones.

Thus, the present invention is primarily based on the technical objective to provide a method to produce a gene library, which avoids the above-mentioned problems.

This technical objective was achieved by providing the embodiments, which are characterized in the claims.

It has surprisingly been found, that a gene library can be produced by means of the method described in the following example, whereat this gene library offers a series of major advantages in comparison to the common methods, in particular in respect to the generation of cDNA libraries, which are optimized for the investigation of gene expression by means of microarrays. The characteristics of a collection of clones produced by the method according to the invention thus prove to be advantageous both in the production of the cDNA-microarrays itself and in the actual employment of the microarrays in hybridization experiments.

Since the cDNA-inserts of the recombinant bacterial clones normally have to be amplified by the polymerase chain reaction (PCR) for producing the cDNA-microarrays, high and homogeneous yields in the PCR are favorable. The amplified products should furthermore be utmostly free of contaminations by other amplified elements, which are due to a non-specific annealing of the primer during PCR. For this reason, the method according to the invention employs specific primer binding sites being ligated at flanking positions of the cDNA-fragments, whereat the sequences of these primer binding sites show an increased GC-contents, which allows to increase the primer binding temperature (annealing temperature) in the PCR. The rise of non-specific background products is significantly less probable in case of using increased annealing temperatures.

In comparison to other cDNA libraries used for microarray applications, the cDNA libraries obtainable by the method according to invention provide the additional advantage, that a very homogeneous size distribution of about 200-600 bp can be achieved, which is not at least also responsible for the homogeneous yields in PCR amplification. Moreover, the homogeneous size distribution of the cDNAs—when producing microarrays at a given concentration of the initial solution—allows to apply nearly equimolar amounts e.g. on a glass carrier. A laborious individual adjustment of the molar concentration of the spotting solution is thus avoided.

A further advantage of the cDNA-fragments obtainable by the method according to the invention, which both affects the production of microarrays and the hybridization results, is the directed/orientated cloning of the cDNAs. In the vector, these cDNAs are present in a uniformly defined orientation, such that—by means of specific PCR-primer modification and suitable coupling chemistry—the “sense strand” alone can be selectively coupled to the employed glass carrier. In standard hybridization with “antisense”-labeled cDNA, such “single strand”-microarrays provide a significantly improved sensitivity in respect to their binding behavior.

A whole series of further advantages directly refers to the behavior of the cDNA-fragments contained within the gene libraries according to the invention when used in complex hybridization for elucidating the level of gene expression. Background hybridizations, which reduce the sensitivity of the system, or non-specific hybridizations, which feign false positive results, are not desired and may negatively influence the test result in case of using inappropriate cDNA-fragments. For this reason, the primer binding sites for PCR amplification of the cDNAs directly flank the cDNA-insert in the method according to the invention. Thus, no non-specific vector parts, which could give reason for an undesirable background hybridization, are amplified; The previous collections of cDNA-clones frequently lead to the generation of PCR products containing larger portions (>100 bp) of vector sequences and thus reduce the specificity of the cDNA amplification product.

A major advantage of the method according to the invention moreover is the generation of cDNA-fragments, which are originally derived from the 3′-untranslated region of the mRNA. These sequence sections mostly develop in a very divergent manner due to the missing selective pressure in phylogenesis, such that even closely related members of a gene family can be clearly distinguished in a hybridization analysis for gene expression. Cross-hybridizations between evolutionary strongly conserved sequence sections preferably occur in the coding sequence portions in conventional cDNA-libraries. Due to the 3′-selection of cDNAs in combination with the stringent size selection of 200-600 bp, primarily the 3′-untranslated regions of transcripts are represented in the cDNA libraries obtainable by the method according to the invention, which permit an increased specificity of hybridization in microarray applications. The homopolymeric poly(A)-tails marking the 3′-end of a transcript, which are frequently present in conventional cDNA libraries, are removed in the course of the method according to the invention. Poly(A)-tails of larger sizes on a microarray reduce the specificity of hybridization and thus cause a deterioration of the signal to noise ratio.

The method according to the invention is mainly based on the following steps:

(a) Starting from mRNA, synthesizing the first cDNA-strand by using a primer, which provides at its 5′-end a first partner of a binding pair, preferably bound in a covalent form, whereat this first binding partner has an affinity to a second binding partner of the binding pair. Suitable binding pairs are familiar to the expert and comprise e.g. biotin/streptavidin, biotin/avidin, antigen/antibody, etc. The primer moreover contains at its 3′-end a poly(dT)-sequence of sufficient length, which is complementary the poly(A)-tail of the mRNA, and—between the 5′-end being coupled to the first binding partner and the poly(dT)-sequence—a sequence, which as a double strand provides a recognition site for a type II restriction enzyme (e.g. BpmI), thereby allowing to later cut off the poly(dT)-sequence or a part thereof from the cDNA. Preferably, the primer is a mixture of primers, which provide the sequence element 5′-VN-3′ at the 3′-side of the poly(dT)-sequence, whereat V is A, C or G and N is A, C, G or T;

(b) second strand synthesis under common conditions, e.g. by (1) degrading the RNA of the DNA/RNA-hybrid with RNAse H, (2) generating the complementary strand with DNA-Polymerase I and (3) linking the open Okazaki-fragments by means of ligase;

(c) fragmentation of the DNAs of step (b) by means of common methods, preferably by using random fragmentation, whereat fragmentation by means of sonication is preferred;

(d) isolation of fragments with the desired range of length by means of common methods if favorable, e.g. by using agarose gel electrophoresis and isolating the desired fragments from the gel. Preferably isolated are fragments in the range of 100 to 1000 bp, more preferred in the range of 200 to 600 bp. In the following, the ends of the DNA-fragments are preferably enzymatically transformed into blunt ends, i.e. the overhangs of the 3′-end or of the 5′-end are removed, for example by using Pwo-DNA-Polymerase or Pfu-DNA-Polymerase;

(e) binding of the fragments, which correspond to the 3′-region of the mRNA to the second binding partner via the first binding partner, whereat the second binding partner is coupled to a solid carrier. Suitable solid carriers are familiar to the expert; they comprise e.g. paramagnetic pearls. The fragments, which remain unattached (fragments without the first binding partner) are then removed by washing.

(f) an adaptor molecule is ligated to the DNA-fragment end opposing the biotinylated end of the DNA-fragment by means of common methods. This adaptor in its 5′-overhanging sequence contains a first restriction site for a restriction enzyme, which preferably cleaves in such a way, that DNA-ends with sequence overhangs are generated (to this: also see the exemplified adaptor in the following example);

(g) a PCR is performed with the fragments still being coupled to the solid carrier, whereat the following primers are used: The 5′-primer is a primer, which mainly corresponds to the sequence of the overhanging adaptor end (or a part thereof). The 3′-primer at its 5′-end is connected to a first binding partner of a binding pair, which can be identical with or different to the binding partner of step (a), and has a sequence mainly corresponding to the sequence of the primer used for first strand synthesis including the restriction site for a type II restriction enzyme. Thus, the 3′-primer in the PCR causes the synthesis of a strand, the sequence of which largely corresponds to that of the first cDNA-strand. The 5′-primer anneals to this strand, thereby leading to the synthesis of the complementary strand under introduction of the restriction site of the adaptor;

(h) after termination of the PCR and preferable purification of the amplified products, a digest is performed with the suitable type II restriction enzyme, whereby the poly(dT)-tails are cut off. Preferably, two to four dT-residues are retained at the fragments. The cleaved poly(dT)-tails are then removed from the fragments now being without a binding partner by binding via their first binding partner to the second binding partner, which is coupled to a solid carrier;

(i) the PCR-fragments disconnected from the poly(dT)-tails (PCR-fragments without binding partner) are preferably again tested in respect to their length and fragments within the desired range of length (see step d) are isolated. Thereafter, an adaptor is ligated to that fragment end, the poly(dT)-tail of which has been removed. This adaptor preferably provides a free d(TT) at one end and is thus able to hybridize to the d(AA)-overhang of the preferred PCR-fragments of step (h). At its other overhanging end, the adaptor provides a recognition site for a second restriction enzyme, which preferably produces ends with sequence overhangs, and whereat the recognition site preferably is different from the first recognition site of the first adaptor molecule in step (f); for a possible design of this adapter we refer to the following example; and in the last step the fragments of step (i) are cut with the restriction enzyme for the first recognition site (the restriction cleavage site for the second restriction enzyme is already “open”) and the fragments are ligated into a vector, which has been cut in a parallel manner. Preferably, the fragments are inserted by ligation in a defined orientation. It is not obligate, that this vector is already the vector used for the actual gene library, but it may also be an “intermediate” vector. By means of this vector, the ligation mixture is amplified in an appropriate host, e.g. E. coli by means of standard protocols (see e.g. Maniatis et al., 1989, “Molecular Cloning: A Laboratory Manual”, Cold Spring Harbor Laboratory, Cold Spring Harbor, N.Y.). After the in vivo-amplification, the vector-DNA is isolated from the transformants and introduced into the final host in order to establish the final gene library (see e.g. the review article in “Current Protocols in Molecular Biology”, second ed., 1988, eds.: Ausubel et al., Greene Publish Assoc. & Wiley Interscience, chapter 13).

Thus, the present invention refers to a method for producing a gene library, whereat said method comprises the following steps:

a) Production of a double-stranded cDNA from a mRNA-population, wherein a specific primer is used for the synthesis of the first cDNA-strand, this primer providing at its 5′-end a first binding partner of a binding pair with an affinity to a second binding partner of the binding pair, this primer further containing at its 3′-end a poly(dT)-sequence, which is complementary the poly(A)-tail of the mRNA, and furthermore containing a sequence, which as a double strand comprises a recognition site for a type II restriction enzyme, whereat said sequence is located between the 5′-end coupled to the first binding partner and the poly(dT)-sequence;

b) fragmentation of the cDNA obtained in step (a);

c) if desired, isolation of fragments of step (b) having the desired length;

d) if desired, providing the (isolated) fragments with blunt ends;

e) binding the fragments of the respective previous step to a second binding partner, which is coupled to a solid carrier;

f) ligating a double-stranded adaptor molecule to the fragment end opposing the end connected to the first binding partner, whereat the adaptor molecule contains a restriction site for a second restriction enzyme;

g) Performance of an in vitro-amplification of the fragments being bound to the carrier by means of a pair of primers, whereat the first primer of the primer pair is mainly complementary to a strand of the adaptor molecule, and whereat the second primer of the primer pair at its 5′-end provides a first binding partner with an affinity to a second partner of a binding pair, and the sequence of which mainly corresponds to the sequence of the primer used in step (a) or a sequence complementary thereto;

h) if desired, cleaving the amplification products of step (e) with a type II restriction enzyme, which recognizes the restriction site introduced in step (a);

i) if desired, ligating an adaptor molecule to the 5′-end of the PCR-products obtained after cleaving with the type II restriction enzyme according to the previous step;

j) if desired, cleaving the products of step (i) by means of a second restriction enzyme, which recognizes the restriction site introduced in step (f), whereat the steps (i) and (j) may also be performed in a reverse order; and

k) ligating the products of the respective previous step into a vector.

The chronological order of single or several steps of the method according to the invention does not have to be performed categorically in the presented order, but may potentially also be varied. Moreover, the mentioned steps c), d) and h)-j) are to be understood as optional. The production of the first and second cDNA-strand is accomplished by means of standard methods under employment of common enzymes and buffers. The expert also is familiar with suitable methods for the isolation of RNA from a sample, wherein the mRNA preferably is enriched e.g. by means of an oligo(dT)-column. The length of the poly(dT)-sequence of the primers used for the first strand synthesis is not critical; it usually ranges between 15 to 30 residues, whereat 18-mers to 21-mers are preferred. Preferably used for the first strand synthesis is a mixture of primers, which provide the sequence 5′-VN-3′ at the 3′-side of the poly(dT)-sequence, whereat V can be A, C or G and N can be A, C, G or T.

The expert also knows about suitable binding partners of the binding pair, e.g. antigen/antibody, antibody/digoxigenin, biotin/avidin and biotin/streptavidin, whereat biotin/avidin and biotin/streptavidin are preferred.

The recognition site of the type II restriction enzyme may be any recognition site of a type II restriction enzyme, e.g. BpmI, AlwI, BpsI, BpvI, Bci VI, BsaI, Bse RI, BsgI, Bsm A1, Earl, EciI, FokI, HgaI, HphI, MboII, PleI, SapI and SfaNI, whereat BmpI is preferred.

The fragmentation of the synthesized cDNAs is such accomplished, that the most fragments possible are obtained in a desired range of size. Suitable techniques of fragmentation are familiar to the expert, whereat a random fragmentation is preferred. Particularly preferred is a fragmentation by means of sonication, whereat the expert can determine—by a series of tests preceding a preparative fragmentation and by variation of parameters like duration of acoustic irradiation and/or intensity within these tests—the respective conditions, which lead to a fragmentation in the desired range of size. Also suitable for fragmentation according to the invention are enzymatic digests with restriction enzymes recognizing 4-mer, 6-mer or 8-mer sequence elements.

The isolation of fragments in the desired range of length under removal of shorter or longer fragments can—if desired—be accomplished by means of any method, in which nucleic acid fragments are separated according to their length, preferably under non-denaturating conditions, e.g. by means of gel electrophoresis, capillary gel electrophoresis, selective precipitation with ammonium acetate/EtOH, whereat gel electrophoresis is preferred. Particularly preferred is agarose gel electrophoresis. During or after the termination of gel electrophoresis, the bands can e.g. be stained, excised and isolated from the gel by means of common methods.

In a preferred embodiment of the method according to the invention, the cDNA-fragments have a length in the range between 100 to 1000 bp, more preferably in the range between 200 to 600 bp.

The expert also knows about methods allowing to provide the isolated cDNA-fragments—the ends of which may have overhangs after the fragmentation—with blunt ends. This can be accomplished by means of common enzymatic methods, whereat however only those strategies are suitable for the method according to the invention, which do not alter the poly(dA)/(dT)-end of the fragments connected to the first binding partner. A suitable method e.g. is the treatment with Pwo-DNA-Polymerase or Pwo-DNA-Polymerase. In dependence on the fragmentation method used, the blunt-end-formation may also be abandoned. If one e.g. uses enzymatic fragmentation, blunt-end-formation is not necessary, since the adaptor of step f) can also be ligated “strictly”.

In the method according to the invention, the second binding partner is preferably bound to a solid carrier, e.g. a microscopic slide, a microtiter plate, pearls consisting of organic (e.g. agarose or polyacrylamide) or inorganic compounds, etc, whereat paramagnetic pearls are preferred. The expert is also familiar with methods for coupling the second binding partner to the solid carrier. The expert furthermore is familiar with conditions—according to the binding pair chosen—which allow to bind the cDNA-fragments to the second binding partner via their first binding partner and to remove the non-bound fragments from the mixture.

Suitable double-stranded adaptor molecules and methods for blunt-end-ligation of the blunt ends of the adaptor molecules to the blunt ends of the cDNA-fragments are known to the expert. The adaptor molecule comprises a recognition site for a restriction enzyme, whereat this restriction enzyme preferably is a restriction enzyme producing overhanging ends, and the recognition site of which extends to at least 6 bp, preferably 8 bp, in order to exclude or largely decrease the probability, that an undesired cleavage within the cDNA-fragments will occur at a later stage of the method according to the invention. Preferred restriction enzymes are AscI, NotI, SrfI, PacI, PmeI, SwaI as well as further restriction enzymes known to the expert, which recognize a 6-mer or 8-mer sequence. The cDNA-fragments bound to the solid carrier are preferably used to perform an in vitro-amplification in order to obtain sufficient amounts of material for the following process steps. Suitable in vitro-amplification methods are well known to the expert, whereat PCR is one preferred amplification method. The primer sequences, which are largely complementary to a strand of the adaptor molecule or to a sequence of the primer of step (a) or to a sequence complementary thereto, have anyhow to be chosen that way, that one primer is complementary to the end of the extended synthesis product of the other primer, thereby permitting exponential amplification.

In a particularly preferred embodiment of the method according to the invention, the cleavage by means of a restriction enzyme in step (h) is such accomplished, that the obtained fragment provides a single-stranded end with at least 2 adenine residues and whereat the adaptor molecule in step (i) preferably provides an overhanging end with the respective dT-residues complementary thereto. A fragment with two overhanging adenine residues is e.g. obtained by a digest with BmpI.

At the end of the method according to the invention, the ligation—preferably a directed ligation —of the cDNA-fragments into a suitable vector is accomplished. Examples of such vectors are known to the expert. Preferably, the aforementioned DNA-fragments are cloned into an expression vector. In case of an expression vector for E. coli, these are e.g. exemplified by pGEMEX, derivatives of pUC (e.g. pUC8), pBR322, pBlueScript, pGEX-2T, pET3b and pQE-8. Expression vectors in yeast are exemplified by pY100 and Ycpad1, whereas e.g. pKCR, PEFBOS, cDM8 and pCEV4 are to be mentioned for the expression in animal cells. Particularly suitable for the expression in insect cells is the baculovirus-expression vector pAcSGHisNT-A. If one desires no expression, it is also possible to clone into the vectors in a manner contrary to the frame. Within the vectors, the DNA-fragments are functionally linked to regulatory elements, which permit their expression in prokaryotic or eukaryotic host cells. Such vectors besides the regulatory elements (e.g. a promoter) also typically contain an origin of replication and specific genes, which allow the transformed host cells to be selected in dependence on a phenotype. Examples of regulatory elements for the expression in pro-karyotes, e.g. in E. coli, are the lac-, trp- or T7-promotor; respective regulatory elements for the expression in eukaryotes are the AOX1-promotor or the GAL1-promotor for yeast and the CMV-, SV40, RVS-40-promotor, CMV-enhancer or SV40-enhancer for the expression in animal cells. Further examples of suitable promoters are the metallothionein I—and the polyhedrin-promoter. In particular the expression vectors based on T7 are among the suitable vectors for the expression in bacteria (Rosenberg et al., Gene 56 (1987), 125) as well as pMSXND for the expression in mammalian cells (Lee and Nathans, J. Biol. Chem. 263 (1988), 3521). The introduction of the aforementioned vectors into the host cell can be accomplished by means of common methods, e.g. by means of calcium phosphate transfection, DEAE-Dextran-mediated transfection, by means of a transfection mediated by cationic lipids, by means of electroporation, infection, gene gun, etc. These methods are described in standard works of molecular biology.

The present invention also relates to a gene library that can be obtained by the method according to the invention. In a preferred embodiment, the gene library according to the invention is characterized in that at least one sequence of a gene or of a part of a gene is present, which encodes for a protein, which takes part in one of the following processes: amino acid synthesis, cellular metabolism, energy metabolism, fatty acid- and phospholipid-metabolism, purine-, pyrimidine-, nucleoside- and nucleotide-synthesis and —degradation, DNA-replication, transcription, translation, protein transport or protein binding.

In a more preferred embodiment the gene library according to the invention is characterized in that sequences of at least 50 genes or parts thereof, preferably of at least 500 genes or parts thereof and more preferred of at least 800 genes or parts thereof are present, the products of which are involved in the same or various of the afore mentioned processes.

The gene library according to the invention is characterized in that at least 95% of the sequences of the present genes have a length between 200 and 600 base pairs. The expert can achieve such a composition of the gene library by means of suitable methods, e.g. by the isolation of fragments according to these sizes in the intermediate steps of the afore mentioned method, e.g. by agarose gel electrophoresis and elution of fragments in the range of 200 to 600 bp from the gel in accordance with standard protocols.

In a further preferred embodiment, the gene library according to the invention is characterized in that the genes are derived from mouse, rat, dog, human, pig, hamster or cow. Suitable sources for these genes are familiar to the expert.

In an even more preferred embodiment, the gene library according to the invention is characterized in that (a) at least 60%, preferably at least 80% and yet more preferred at least 90% of the sequences comprise genes or parts of genes, which are derived from the 3′-region of the mRNA or that (b) at least 60% of the genes or of the parts thereof, preferably at least 80% and yet more preferred at least 90% comprise sequences without a poly(A)-tail.

Yet more preferred is a gene library according to the invention, which is characterized in that the sequences of the genes or of the parts thereof are present in a prokaryotic plasmid. In respect to suitable plasmids, we refer to the preceding embodiments.

In an even more preferred embodiment, the gene library according to the invention is characterized in that sequences of at least 50 genes or of parts thereof, preferably of 200 genes or of parts thereof and most preferred of 500 genes or of parts thereof are present, which are selected from:

(a) the sequences of the SEQ ID-list “Replication”,

(b) the sequences of the SEQ ID-list “Transcription”,

(c) the sequences of the SEQ ID-list “Translation”,

(d) the sequences of the SEQ ID-list “Transport- and binding Proteins”, or

(e) combinations of at least two of the groups (a) to (d).

The distinct sequences are given in the sequence protocol, which comprises the SEQ ID No. 1 to SEQ ID No. 840 and is an integral part of the patent application.

The present invention also refers to transformants containing the previously described gene library. These transformants encompass bacteria, yeast, insect—and animal cells, preferably mammalian cells. Favorable hosts are the E. coli strains HB101, DH1, DH10B, x1776, JM101, JM109, BL21, XL1Blue and SG 13009, the yeast species Saccharomyces cerevisiae and the animal cells L, 3T3, FM3A, CHO, COS, Vero, HeLa and the insect cell line sf9. Methods for transforming these host cells, methods for phenotypical selection of transformants and for expressing the DNA-sequences comprised within the gene library under employment of the above described vectors are known in this special field.

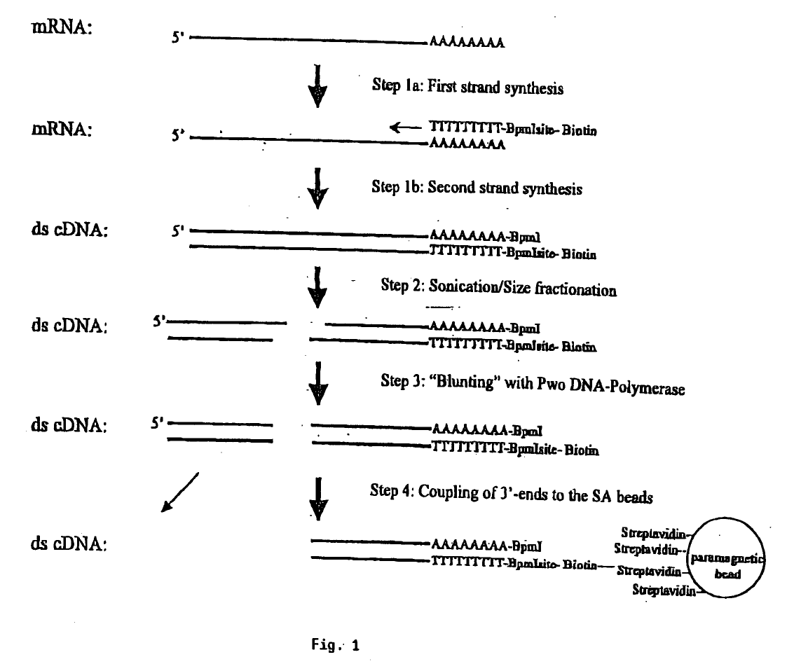

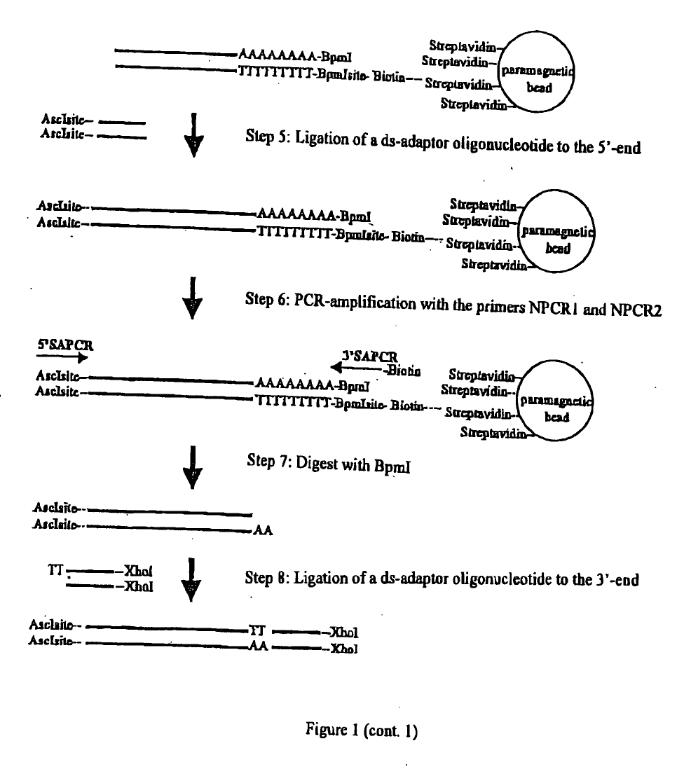

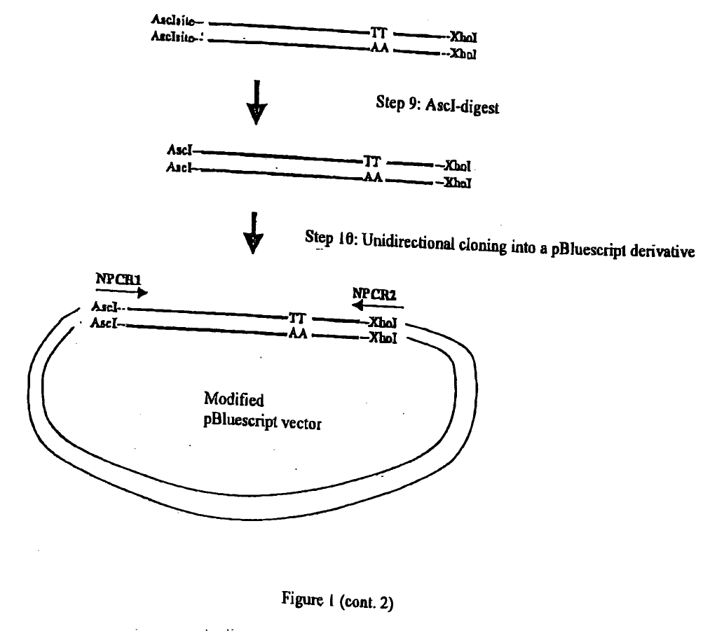

FIG. 1: Schematic depiction of the distinct steps for producing a cDNA gene library as described in the following example

The following example illustrates the invention.

### EXAMPLE

### Production of a Gene Library From the mRNA of Mouse Liver

(A) Preparation of mRNA

The RNA was isolated from homogenized mouse liver by using a kit for RNA-purification (Trizol, Life Technologies, Rockville, USA), followed by employing a kit for mRNA-isolation (Dynabeads mRNA Purification Kit, Dynal A. S., Oslo, Norway) in accordance with the manufacturer's recommendations. A total of about 2 μg of mRNA was used to construct the gene library.

(B) Synthesis of a Double-Stranded cDNA

The synthesis of the cDNA-first strand by means of the Gibco “cDNA synthesis system” (Fa. GibcoBRL Life Technologies GmbH, Karlsruhe; Cat.No. 18267-013) was accomplished in accordance with the manufacturer's information by using the permutated primer 5′-ATG ATG CTG GAG TTT TTT TTT TTT TTT TTT VN-3′, whereat V is A, C or G and N is A, C, G or T. The primer additionally carried a biotin residue at its 5′-end. The nucleotides underlined represent a BpmI-restriction site (type II restriction enzyme). After degradation with RNAse H, DNA-polymerase I was used for second strand-synthesis and ligase was used for connecting the open Okazaki-fragments according to common conditions. In the following, the generated double-stranded DNA was precipitated with ethanol, dried and resuspended in buffer.

(C) Fragmentation of the cDNA by Means of Sonication

The cDNA was treated with ultrasound for 1 minute with a pulse of 0.9 by using the Misonex 2020 System (Fa. Misonix, Farmingdale, N.Y., USA). The successful sonication was verified by gel electrophoresis of an aliquot. After positive control of the aliquots, the whole reaction mixture was separated by means of agarose gel electrophoresis and fragments in the size range corresponding to the insert length of 200 to 600 bp, which is desired for the gene library to be produced, were eluted from the agarose gel by means of a “QIAquick gel extraction kit (Qiagen).

(D) Providing the Eluted cDNA-Fragments With Blunt Ends

After the sonication, the isolated DNA-fragments also provide non-specific 5′- or 3′-overhanging ends. These ends were filled or cleaved with Pwo-DNA-polymerase (Hoffman LaRoche, Basel, Switzerland) in order to generate blunt ends. To this aim, 30 μl of DNA-solution were mixed with 8 μl of a dNTP-mixture (200 μM per dNTP), 2 μl distillated water, 4.5 μl 10×Pwo-buffer (with MgSO₄) and 0.5 μl (5 E/μl) Pwo-DNA-polymerase, followed by an incubation at 70° C. for 30 minutes. The reaction was then stopped on ice.

(E) Coupling the 3′-Ends to Streptavidin-Coated Paramagnetic Pearls (“SA-beads”)

Via their biotinylated 5′-end (corresponding to the 3′-end of the mRNA), the fragments were bound by the non-biotinylated fragments (corresponding to internal or 5′-regions of the mRNA) by binding to paramagnetic pearls (Dynabeads M-280 Streptavidin; Fa. Dynal, Oslo, Norway) according to the manufacturer's information. By means of the following washing step, the remaining fragments (without biotinylation) were removed.

(F) Ligation of a Double-Stranded Adaptor-Oligonucleotide

In the following, a double-stranded adaptor-oligonucleotide with an internal AscI-restriction site and the following sequence of 5′-CTA ATA CGA CTC ACT ATA GGG CGC GCC AGC GTG GTC GCG GCC GAG GT-3′; 3′-CAG CGC CGG CTC CA-5′, was blunt-end ligated under standard conditions to the fragment end opposing the biotinylated end of the DNA-fragments, whereby the DNA-fragments are still bound to the pearls. In this adaptor, the underlined sequence corresponds to the T7-promotor sequence, the sequence GGCGCGCC printed in boldface corresponds to the 5′-restriction site of AscI, which is used for cloning into the plasmid vector, and the sequence ACCTCGGCCGCGAC corresponds to the complementary “auxiliary” oligonucleotide. The reaction was performed overnight at 16° C. in a volume of 20 μl. The reaction mix contained 1 μl of the coupled DNA, 2 μl of T4-DNA-ligase (40 E/μl; Roche) and 5 μl of the double-stranded adaptor-oligonucleotide (final concentration: 2.5 μM).

(G) Direct PCR-Amplification of the 3′-Ends at the “SA-Beads”

In order to obtain sufficient material for the following steps of the process, the fragments still being bound to the paramagnetic pearls and corresponding to the 3′-regions of the transcripts were amplified by means of PCR, whereat the following primers were used: 5′-PCR-primer (5′ SAPCR): 5′-CTA ATA CGA CTC ACT ATA GGG C-3′; 3′-primer (3′ SAPCR): biotin-5′-ATG ATG CTG GAG TTT TTT TTT TTT TTT T-3′. Thereby, the 5′-PCR primer lies on the ligated adapter, whereas the biotinylated 3′-PCR primer lies on the first strand synthesis-primer (including the BpmI-restriction site). First, the optimal number of PCR-cycles was determined for an aliquot of the reaction in order to later use this number of cycles for the preparative approach. Thereby, 10 μl of the ligation mixture were used as a template, 2 μl Pwo-DNA-polymerase (Roche), 1 μl of both amplification primers 5′ SAPCR (10 μM) and 3′ SAPCR (10 μM) each, and 10 μl of dNTP-solution (concentration per dNTP: 1 mM). The reaction was performed in a volume of 50 μl in a DNA Thermal Cycler (GeneAmp PCR Systems 9700; Perkin Elmer Applied Biosystems, Weiterstadt). After an initial step of denaturation (5 min, 75° C.; 30 sec, 94° C.), a total of 30 cycles with the following profile was performed: 10 seconds at 94° C., 30 seconds at 60° C. and 90 seconds at 72° C. In the following, the process terminates with a final step of elongation for 5 minutes at 72° C. After the PCR-reaction, the PCR-products were purified by means of the “Quiaquick PCR Purification Kit” (Qiagen).

(H) Digest of the PCR-Products with the Restriction Enzyme BpmI (type IIS Enzyme)

In the next step, a restriction digest was performed by using the restriction enzyme BmpI (NEB, Beverly, Mass., USA). Since a BpmI-restriction site has been incorporated during the first strand synthesis (see (A)) in direct neighborhood to the poly(T)-tail of the cDNA, the restriction digest at the one hand releases the cDNA-molecules from the “SA-Beads”, and at the other hand—and except for two T-residues—cuts off the poly(T)-tails of the PCR-products from those ends of the DNA-fragments corresponding to the 3′-ends of the transcripts. The “SA-Beads” still carrying the poly(T)-tails are then separated from the cDNA-fragments, which correspond to the 3′-ends of the mRNA, and which are now present in a free form in the solution. The fragments were again loaded onto a 1% agarose gel and fragments having a length between 200 bp and 600 bp were excised and eluted as described above.

(I) Ligation of a Double-Stranded Adaptor Molecule to the cDNA-Molecules Corresponding to the 3′-Ends of the mRNA

In the next step, a 3′-TT-adaptor was ligated at the released fragment end corresponding to the 3′-end of the mRNA. This adapter provides an “open” XhoI-restriction site at its 3′-end. This adaptor had the following sequence: 5′-TCG AGC GGC CGC CCG GGC AGG TTT-3′; 3′-CG CCG GCG GGC CCG TCC A-5′. This adaptor in its left portion contains an open XhoI-restriction site and in its right portion a single-stranded overhanging end with TT, which is compatible to the 3′-overhanging AA of the cDNAs. The lower adaptor sequence is phosphorylated at its 5′-end. The reaction was performed overnight at 16° C. in a volume of 100 μl, whereat 30 μl of DNA-solution, 2 μl of T4 DNA-ligase (400 E/μl; Roche) and 20 μl of the double-stranded adaptor-DNA (210 μM) were used. When annealing the two complementary adaptor oligonucleotides, two overhanging TT-nucleotides are produced at one site—these nucleotides permitting a ligation to the BpmI-digested cDNA—whereas at the other site an open XhoI-restriction site is created, which is later used for the unidirectional cloning.

(J) Restriction Digest of the cDNA-Fragments with AscI

In the following, the AscI-restriction site of the ligated fragments obtained that way was opened by cleavage with AscI (NEB). The fragments thereafter carried an AscI-restriction site at their 5′-end and a XhoI-restriction site at their 3′-end. This was followed by another validation of the correct size by means of agarose gel electrophoresis, whereby fragments with the desired insert length (200 to 600 bp) were eluted from the gel as described above.

(K) Unidirectional Cloning

The eluted fragments were finally ligated in an orientated manner into a BlueScript derivative (pBSC-NTER; Fa. Stratagene, Heidelberg, Germany), which was such modified, that it contained within its polylinker the restriction sites compatible to the two restriction sites of the cDNA-fragments and thus allowed to be digested with the respective restriction enzymes. This was followed by a transformation into competent DH10B-cells (GibcoBRL Life Technologies GmbH, Karlsruhe). As a control, the cDNA-inserts were amplified with the PCR-primers “NPCR1” (5′-TCG AGC GGC CGC CCG GGC AGG T-3′) and “NPCR2” (5′-AGC GTG GTC GCG GCC GAG GT-3′), which are lying on the two adaptors. In this amplification, no additional parts of the vector were co-amplified.

SEQ ID-List “Replication”

| | | |
|---|---|---|
| SEQ ID No 25 | MOUSE: MG-13-4c19 | Replication |
| SEQ ID No 26 | MOUSE: MG-13-4e3 | Replication |
| SEQ ID No 46 | MOUSE: MG-14-4e9 | Replication |
| SEQ ID No 53 | MOUSE: MG-15-1e3 | Replication |
| SEQ ID No 65 | MOUSE: MG-16-3b14 | Replication |
| SEQ ID No 69 | MOUSE: MG-16-4c5 | Replication |
| SEQ ID No 79 | MOUSE: MG-16-6b20 | Replication |
| SEQ ID No 85 | MOUSE: MG-16-7a13 | Replication |
| SEQ ID No 92 | MOUSE: MG-16-7m2 | Replication |
| SEQ ID No 98 | MOUSE: MG-16-9b6 | Replication |
| SEQ ID No 100 | MOUSE: MG-16-9m14 | Replication |
| SEQ ID No 104 | MOUSE: MG-19-2c13 | Replication |
| SEQ ID No 112 | MOUSE: MG-3-100b4 | Replication |
| SEQ ID No 118 | MOUSE: MG-3-106m10 | Replication |
| SEQ ID No 119 | MOUSE: MG-3-106n16 | Replication |
| SEQ ID No 122 | MOUSE: MG-3-108j10 | Replication |
| SEQ ID No 123 | MOUSE: MG-3-108j3 | Replication |
| SEQ ID No 160 | MOUSE: MG-3-12a9 | Replication |
| SEQ ID No 161 | MOUSE: MG-3-12e6 | Replication |
| SEQ ID No 169 | MOUSE: MG-3-13k7 | Replication |
| SEQ ID No 174 | MOUSE: MG-3-142o11 | Replication |
| SEQ ID No 182 | MOUSE: MG-3-15a14 | Replication |
| SEQ ID No 183 | MOUSE: MG-3-15f4 | Replication |
| SEQ ID No 187 | MOUSE: MG-3-18j5 | Replication |
| SEQ ID No 190 | MOUSE: MG-3-1e22 | Replication |
| SEQ ID No 203 | MOUSE: MG-3-22m14 | Replication |
| SEQ ID No 209 | MOUSE: MG-3-23j6 | Replication |
| SEQ ID No 216 | MOUSE: MG-3-25a15 | Replication |
| SEQ ID No 217 | MOUSE: MG-3-25d11 | Replication |
| SEQ ID No 220 | MOUSE: MG-3-25i17 | Replication |
| SEQ ID No 221 | MOUSE: MG-3-25k21 | Replication |
| SEQ ID No 225 | MOUSE: MG-3-26l10 | Replication |
| SEQ ID No 241 | MOUSE: MG-3-2c8 | Replication |
| SEQ ID No 244 | MOUSE: MG-3-2k6 | Replication |
| SEQ ID No 250 | MOUSE: MG-3-31m18 | Replication |
| SEQ ID No 268 | MOUSE: MG-3-37n1 | Replication |
| SEQ ID No 276 | MOUSE: MG-3-3i15 | Replication |
| SEQ ID No 277 | MOUSE: MG-3-3o3 | Replication |
| SEQ ID No 282 | MOUSE: MG-3-43c4 | Replication |
| SEQ ID No 285 | MOUSE: MG-3-43k7 | Replication |
| SEQ ID No 290 | MOUSE: MG-3-44m16 | Replication |
| SEQ ID No 291 | MOUSE: MG-3-45k11 | Replication |
| SEQ ID No 297 | MOUSE: MG-3-46o2 | Replication |
| SEQ ID No 300 | MOUSE: MG-3-47l23 | Replication |
| SEQ ID No 310 | MOUSE: MG-3-49h15 | Replication |
| SEQ ID No 313 | MOUSE: MG-3-4c14 | Replication |
| SEQ ID No 354 | MOUSE: MG-3-64k13 | Replication |
| SEQ ID No 367 | MOUSE: MG-3-71n17 | Replication |
| SEQ ID No 379 | MOUSE: MG-3-75j6 | Replication |
| SEQ ID No 380 | MOUSE: MG-3-75n23 | Replication |
| SEQ ID No 382 | MOUSE: MG-3-76b11 | Replication |
| SEQ ID No 399 | MOUSE: MG-3-7n12 | Replication |
| SEQ ID No 401 | MOUSE: MG-3-80h21 | Replication |
| SEQ ID No 402 | MOUSE: MG-3-80l14 | Replication |
| SEQ ID No 405 | MOUSE: MG-3-81h2 | Replication |
| SEQ ID No 415 | MOUSE: MG-3-85f14 | Replication |
| SEQ ID No 420 | MOUSE: MG-3-88k7 | Replication |
| SEQ ID No 425 | MOUSE: MG-3-8f8 | Replication |
| SEQ ID No 438 | MOUSE: MG-3-95i1 | Replication |
| SEQ ID No 439 | MOUSE: MG-3-95o9 | Replication |
| SEQ ID No 441 | MOUSE: MG-3-96p21 | Replication |
| SEQ ID No 450 | MOUSE: MG-4-145n21 | Replication |
| SEQ ID No 452 | MOUSE: MG-4-145o24 | Replication |
| SEQ ID No 454 | MOUSE: MG-4-146h14 | Replication |
| SEQ ID No 462 | MOUSE: MG-4-148g1 | Replication |
| SEQ ID No 492 | MOUSE: MG-6-13h17 | Replication |
| SEQ ID No 496 | MOUSE: MG-6-15c24 | Replication |
| SEQ ID No 519 | MOUSE: MG-6-24o2 | Replication |
| SEQ ID No 521 | MOUSE: MG-6-29m3 | Replication |
| SEQ ID No 531 | MOUSE: MG-6-31p16 | Replication |
| SEQ ID No 535 | MOUSE: MG-6-3213 | Replication |
| SEQ ID No 537 | MOUSE: MG-6-33j6 | Replication |
| SEQ ID No 540 | MOUSE: MG-6-35k17 | Replication |
| SEQ ID No 557 | MOUSE: MG-6-3i20 | Replication |
| SEQ ID No 565 | MOUSE: MG-6-40e19 | Replication |
| SEQ ID No 571 | MOUSE: MG-6-41g13 | Replication |
| SEQ ID No 581 | MOUSE: MG-6-43p21 | Replication |
| SEQ ID No 622 | MOUSE: MG-6-64m19 | Replication |
| SEQ ID No 631 | MOUSE: MG-6-6p14 | Replication |
| SEQ ID No 634 | MOUSE: MG-6-71j22 | Replication |
| SEQ ID No 640 | MOUSE: MG-6-75a6 | Replication |
| SEQ ID No 659 | MOUSE: MG-6-82p23 | Replication |
| SEQ ID No 692 | MOUSE: MG-8-117o11 | Replication |
| SEQ ID No 707 | MOUSE: MG-8-12n3 | Replication |
| SEQ ID No 714 | MOUSE: MG-8-13n3 | Replication |
| SEQ ID No 715 | MOUSE: MG-8-14g1 | Replication |
| SEQ ID No 719 | MOUSE: MG-8-16a12 | Replication |
| SEQ ID No 734 | MOUSE: MG-8-20o20 | Replication |
| SEQ ID No 753 | MOUSE: MG-8-27k19 | Replication |
| SEQ ID No 771 | MOUSE: MG-8-33b18 | Replication |
| SEQ ID No 778 | MOUSE: MG-8-34d4 | Replication |
| SEQ ID No 790 | MOUSE: MG-8-38p11 | Replication |
| SEQ ID No 796 | MOUSE: MG-8-40g12 | Replication |
| SEQ ID No 815 | MOUSE: MG-8-51c20 | Replication |
| SEQ ID No 817 | MOUSE: MG-8-52d1 | Replication |
| SEQ ID No 823 | MOUSE: MG-8-54g9 | Replication |

SEQ ID-List “Transcription”

| | | |
|---|---|---|
| SEQ ID No 7 | MOUSE: MG-11-1o21 | Transcription |
| SEQ ID No 21 | MOUSE: MG-13-1g21 | Transcription |
| SEQ ID No 27 | MOUSE: MG-13-4n3 | Transcription |
| SEQ ID No 29 | MOUSE: MG-13-6o14 | Transcription |
| SEQ ID No 52 | MOUSE: MG-14-5j23 | Transcription |
| SEQ ID No 54 | MOUSE: MG-15-2e20 | Transcription |
| SEQ ID No 62 | MOUSE: MG-16-10g4 | Transcription |
| SEQ ID No 63 | MOUSE: MG-16-10o16 | Transcription |
| SEQ ID No 64 | MOUSE: MG-16-2k3 | Transcription |
| SEQ ID No 71 | MOUSE: MG-16-5b5 | Transcription |
| SEQ ID No 73 | MOUSE: MG-16-5i18 | Transcription |
| SEQ ID No 94 | MOUSE: MG-16-8n16 | Transcription |
| SEQ ID No 115 | MOUSE: MG-3-102p7 | Transcription |
| SEQ ID No 116 | MOUSE: MG-3-103m15 | Transcription |
| SEQ ID No 121 | MOUSE: MG-3-108d19 | Transcription |
| SEQ ID No 125 | MOUSE: MG-3-109l17 | Transcription |
| SEQ ID No 126 | MOUSE: MG-3-109p13 | Transcription |
| SEQ ID No 127 | MOUSE: MG-3-10c1 | Transcription |
| SEQ ID No 130 | MOUSE: MG-3-10k3 | Transcription |
| SEQ ID No 134 | MOUSE: MG-3-110e4 | Transcription |
| SEQ ID No 138 | MOUSE: MG-3-113f21 | Transcription |
| SEQ ID No 139 | MOUSE: MG-3-113j5 | Transcription |
| SEQ ID No 140 | MOUSE: MG-3-113m21 | Transcription |
| SEQ ID No 142 | MOUSE: MG-3-114n3 | Transcription |
| SEQ ID No 144 | MOUSE: MG-3-116m5 | Transcription |
| SEQ ID No 151 | MOUSE: MG-3-11l11 | Transcription |
| SEQ ID No 155 | MOUSE: MG-3-122a21 | Transcription |
| SEQ ID No 162 | MOUSE: MG-3-12j14 | Transcription |
| SEQ ID No 163 | MOUSE: MG-3-12j20 | Transcription |
| SEQ ID No 164 | MOUSE: MG-3-12k8 | Transcription |
| SEQ ID No 165 | MOUSE: MG-3-136n16 | Transcription |
| SEQ ID No 168 | MOUSE: MG-3-13k15 | Transcription |
| SEQ ID No 170 | MOUSE: MG-3-140a7 | Transcription |
| SEQ ID No 172 | MOUSE: MG-3-140n23 | Transcription |
| SEQ ID No 176 | MOUSE: MG-3-143h24 | Transcription |
| SEQ ID No 180 | MOUSE: MG-3-14f24 | Transcription |
| SEQ ID No 185 | MOUSE: MG-3-18d4 | Transcription |
| SEQ ID No 186 | MOUSE: MG-3-18h18 | Transcription |
| SEQ ID No 189 | MOUSE: MG-3-19k16 | Transcription |
| SEQ ID No 194 | MOUSE: MG-3-20n20 | Transcription |
| SEQ ID No 197 | MOUSE: MG-3-21n21 | Transcription |
| SEQ ID No 199 | MOUSE: MG-3-22e2 | Transcription |
| SEQ ID No 202 | MOUSE: MG-3-22m1 | Transcription |
| SEQ ID No 206 | MOUSE: MG-3-23d22 | Transcription |
| SEQ ID No 212 | MOUSE: MG-3-24h16 | Transcription |
| SEQ ID No 213 | MOUSE: MG-3-24l17 | Transcription |
| SEQ ID No 215 | MOUSE: MG-3-24o2 | Transcription |
| SEQ ID No 218 | MOUSE: MG-3-25f1 | Transcription |
| SEQ ID No 223 | MOUSE: MG-3-26a2 | Transcription |
| SEQ ID No 228 | MOUSE: MG-3-27d20 | Transcription |
| SEQ ID No 229 | MOUSE: MG-3-27i13 | Transcription |
| SEQ ID No 233 | MOUSE: MG-3-28d3 | Transcription |
| SEQ ID No 235 | MOUSE: MG-3-28i9 | Transcription |
| SEQ ID No 238 | MOUSE: MG-3-29l11 | Transcription |
| SEQ ID No 240 | MOUSE: MG-3-29n9 | Transcription |
| SEQ ID No 242 | MOUSE: MG-3-2d4 | Transcription |
| SEQ ID No 252 | MOUSE: MG-3-32f14 | Transcription |
| SEQ ID No 254 | MOUSE: MG-3-32j16 | Transcription |
| SEQ ID No 255 | MOUSE: MG-3-32k3 | Transcription |
| SEQ ID No 257 | MOUSE: MG-3-33l21 | Transcription |
| SEQ ID No 262 | MOUSE: MG-3-36f22 | Transcription |
| SEQ ID No 264 | MOUSE: MG-3-37h11 | Transcription |
| SEQ ID No 266 | MOUSE: MG-3-37k2 | Transcription |
| SEQ ID No 267 | MOUSE: MG-3-37l19 | Transcription |
| SEQ ID No 270 | MOUSE: MG-3-38m21 | Transcription |
| SEQ ID No 272 | MOUSE: MG-3-38n16 | Transcription |
| SEQ ID No 274 | MOUSE: MG-3-39g12 | Transcription |
| SEQ ID No 275 | MOUSE: MG-3-3f9 | Transcription |
| SEQ ID No 278 | MOUSE: MG-3-40c9 | Transcription |
| SEQ ID No 280 | MOUSE: MG-3-41j5 | Transcription |
| SEQ ID No 281 | MOUSE: MG-3-41p22 | Transcription |
| SEQ ID No 283 | MOUSE: MG-3-43f6 | Transcription |
| SEQ ID No 288 | MOUSE: MG-3-44g1 | Transcription |
| SEQ ID No 294 | MOUSE: MG-3-45p20 | Transcription |
| SEQ ID No 295 | MOUSE: MG-3-46d2 | Transcription |
| SEQ ID No 296 | MOUSE: MG-3-46h17 | Transcription |
| SEQ ID No 303 | MOUSE: MG-3-48k13 | Transcription |
| SEQ ID No 304 | MOUSE: MG-3-48l10 | Transcription |
| SEQ ID No 305 | MOUSE: MG-3-48l24 | Transcription |
| SEQ ID No 307 | MOUSE: MG-3-48o8 | Transcription |
| SEQ ID No 308 | MOUSE: MG-3-49f13 | Transcription |
| SEQ ID No 315 | MOUSE: MG-3-4d16 | Transcription |
| SEQ ID No 322 | MOUSE: MG-3-4n9 | Transcription |
| SEQ ID No 324 | MOUSE: MG-3-50l9 | Transcription |
| SEQ ID No 325 | MOUSE: MG-3-50m3 | Transcription |
| SEQ ID No 326 | MOUSE: MG-3-50n19 | Transcription |
| SEQ ID No 329 | MOUSE: MG-3-51l17 | Transcription |
| SEQ ID No 331 | MOUSE: MG-3-52d14 | Transcription |
| SEQ ID No 333 | MOUSE: MG-3-52p2 | Transcription |
| SEQ ID No 336 | MOUSE: MG-3-55p16 | Transcription |
| SEQ ID No 339 | MOUSE: MG-3-57a6 | Transcription |
| SEQ ID No 342 | MOUSE: MG-3-5c4 | Transcription |
| SEQ ID No 349 | MOUSE: MG-3-62b3 | Transcription |
| SEQ ID No 351 | MOUSE: MG-3-63j4 | Transcription |
| SEQ ID No 355 | MOUSE: MG-3-66b13 | Transcription |
| SEQ ID No 358 | MOUSE: MG-3-66n16 | Transcription |
| SEQ ID No 359 | MOUSE: MG-3-66p18 | Transcription |
| SEQ ID No 368 | MOUSE: MG-3-72g16 | Transcription |
| SEQ ID No 372 | MOUSE: MG-3-74a16 | Transcription |
| SEQ ID No 376 | MOUSE: MG-3-74k7 | Transcription |
| SEQ ID No 378 | MOUSE: MG-3-75e7 | Transcription |
| SEQ ID No 381 | MOUSE: MG-3-75p4 | Transcription |
| SEQ ID No 384 | MOUSE: MG-3-76e9 | Transcription |
| SEQ ID No 387 | MOUSE: MG-3-77h22 | Transcription |
| SEQ ID No 389 | MOUSE: MG-3-78o12 | Transcription |
| SEQ ID No 393 | MOUSE: MG-3-79k7 | Transcription |
| SEQ ID No 395 | MOUSE: MG-3-7f17 | Transcription |
| SEQ ID No 398 | MOUSE: MG-3-7m20 | Transcription |
| SEQ ID No 400 | MOUSE: MG-3-7o16 | Transcription |
| SEQ ID No 403 | MOUSE: MG-3-81f11 | Transcription |
| SEQ ID No 410 | MOUSE: MG-3-84j15 | Transcription |
| SEQ ID No 411 | MOUSE: MG-3-84n2 | Transcription |
| SEQ ID No 412 | MOUSE: MG-3-85b13 | Transcription |
| SEQ ID No 417 | MOUSE: MG-3-87b16 | Transcription |
| SEQ ID No 419 | MOUSE: MG-3-88h12 | Transcription |
| SEQ ID No 421 | MOUSE: MG-3-88l7 | Transcription |
| SEQ ID No 422 | MOUSE: MG-3-88m17 | Transcription |
| SEQ ID No 426 | MOUSE: MG-3-8f9 | Transcription |
| SEQ ID No 429 | MOUSE: MG-3-90e16 | Transcription |
| SEQ ID No 430 | MOUSE: MG-3-90e5 | Transcription |
| SEQ ID No 432 | MOUSE: MG-3-91d6 | Transcription |
| SEQ ID No 435 | MOUSE: MG-3-93m16 | Transcription |
| SEQ ID No 437 | MOUSE: MG-3-94j23 | Transcription |
| SEQ ID No 444 | MOUSE: MG-3-9h15 | Transcription |
| SEQ ID No 449 | MOUSE: MG-4-145l6 | Transcription |
| SEQ ID No 457 | MOUSE: MG-4-146o24 | Transcription |
| SEQ ID No 461 | MOUSE: MG-4-148f6 | Transcription |
| SEQ ID No 465 | MOUSE: MG-4-149d20 | Transcription |
| SEQ ID No 471 | MOUSE: MG-4-2o2 | Transcription |
| SEQ ID No 477 | MOUSE: MG-4-4h6 | Transcription |
| SEQ ID No 479 | MOUSE: MG-4-5b17 | Transcription |
| SEQ ID No 483 | MOUSE: MG-4-5j9 | Transcription |
| SEQ ID No 487 | MOUSE: MG-6-10o24 | Transcription |
| SEQ ID No 488 | MOUSE: MG-6-10o9 | Transcription |
| SEQ ID No 502 | MOUSE: MG-6-16l16 | Transcription |
| SEQ ID No 503 | MOUSE: MG-6-16p8 | Transcription |
| SEQ ID No 504 | MOUSE: MG-6-17n24 | Transcription |
| SEQ ID No 506 | MOUSE: MG-6-19g10 | Transcription |
| SEQ ID No 509 | MOUSE: MG-6-1k5 | Transcription |
| SEQ ID No 514 | MOUSE: MG-6-22n21 | Transcription |
| SEQ ID No 524 | MOUSE: MG-6-2k8 | Transcription |
| SEQ ID No 526 | MOUSE: MG-6-30o15 | Transcription |
| SEQ ID No 527 | MOUSE: MG-6-31b11 | Transcription |
| SEQ ID No 530 | MOUSE: MG-6-31l15 | Transcription |
| SEQ ID No 534 | MOUSE: MG-6-32e14 | Transcription |
| SEQ ID No 538 | MOUSE: MG-6-33o17 | Transcription |
| SEQ ID No 539 | MOUSE: MG-6-35i22 | Transcription |
| SEQ ID No 541 | MOUSE: MG-6-36a15 | Transcription |
| SEQ ID No 542 | MOUSE: MG-6-36b13 | Transcription |
| SEQ ID No 545 | MOUSE: MG-6-36i7 | Transcription |
| SEQ ID No 547 | MOUSE: MG-6-37k24 | Transcription |
| SEQ ID No 566 | MOUSE: MG-6-40g17 | Transcription |
| SEQ ID No 572 | MOUSE: MG-6-41n2 | Transcription |
| SEQ ID No 573 | MOUSE: MG-6-41n6 | Transcription |
| SEQ ID No 575 | MOUSE: MG-6-41o6 | Transcription |
| SEQ ID No 576 | MOUSE: MG-6-42b15 | Transcription |
| SEQ ID No 578 | MOUSE: MG-6-42f2 | Transcription |
| SEQ ID No 583 | MOUSE: MG-6-44a22 | Transcription |
| SEQ ID No 587 | MOUSE: MG-6-46j8 | Transcription |
| SEQ ID No 588 | MOUSE: MG-6-47h20 | Transcription |
| SEQ ID No 590 | MOUSE: MG-6-47o6 | Transcription |
| SEQ ID No 594 | MOUSE: MG-6-48p1 | Transcription |
| SEQ ID No 604 | MOUSE: MG-6-56d22 | Transcription |
| SEQ ID No 609 | MOUSE: MG-6-57j16 | Transcription |
| SEQ ID No 612 | MOUSE: MG-6-59f3 | Transcription |
| SEQ ID No 614 | MOUSE: MG-6-61e6 | Transcription |
| SEQ ID No 616 | MOUSE: MG-6-61k11 | Transcription |
| SEQ ID No 635 | MOUSE: MG-6-71n14 | Transcription |
| SEQ ID No 638 | MOUSE: MG-6-74m9 | Transcription |
| SEQ ID No 642 | MOUSE: MG-6-75e5 | Transcription |
| SEQ ID No 643 | MOUSE: MG-6-75j14 | Transcription |
| SEQ ID No 647 | MOUSE: MG-6-78a4 | Transcription |
| SEQ ID No 663 | MOUSE: MG-6-86g1 | Transcription |
| SEQ ID No 669 | MOUSE: MG-6-89b13 | Transcription |
| SEQ ID No 670 | MOUSE: MG-6-89n5 | Transcription |
| SEQ ID No 671 | MOUSE: MG-6-90a24 | Transcription |
| SEQ ID No 673 | MOUSE: MG-6-90d18 | Transcription |
| SEQ ID No 679 | MOUSE: MG-6-92a3 | Transcription |
| SEQ ID No 689 | MOUSE: MG-8-117m12 | Transcription |
| SEQ ID No 705 | MOUSE: MG-8-12m9 | Transcription |
| SEQ ID No 710 | MOUSE: MG-8-13d9 | Transcription |
| SEQ ID No 718 | MOUSE: MG-8-15l7 | Transcription |
| SEQ ID No 721 | MOUSE: MG-8-16n13. | Transcription |
| SEQ ID No 723 | MOUSE: MG-8-17i1 | Transcription |
| SEQ ID No 728 | MOUSE: MG-8-1e1 | Transcription |
| SEQ ID No 731 | MOUSE: MG-8-1i1 | Transcription |
| SEQ ID No 736 | MOUSE: MG-8-22d8 | Transcription |
| SEQ ID No 740 | MOUSE: MG-8-23c18 | Transcription |
| SEQ ID No 741 | MOUSE: MG-8-23d3 | Transcription |
| SEQ ID No 743 | MOUSE: MG-8-23m17 | Transcription |
| SEQ ID No 744 | MOUSE: MG-8-24g5 | Transcription |
| SEQ ID No 751 | MOUSE: MG-8-26o3 | Transcription |
| SEQ ID No 768 | MOUSE: MG-8-32e6 | Transcription |
| SEQ ID No 775 | MOUSE: MG-8-33f19 | Transcription |
| SEQ ID No 785 | MOUSE: MG-8-36n1 | Transcription |
| SEQ ID No 788 | MOUSE: MG-8-38i4 | Transcription |
| SEQ ID No 793 | MOUSE: MG-8-39p19 | Transcription |
| SEQ ID No 800 | MOUSE: MG-8-44m16 | Transcription |
| SEQ ID No 801 | MOUSE: MG-8-44m23 | Transcription |
| SEQ ID No 804 | MOUSE: MG-8-45c4 | Transcription |
| SEQ ID No 809 | MOUSE: MG-8-48e21 | Transcription |
| SEQ ID No 810 | MOUSE: MG-8-48i10 | Transcription |
| SEQ ID No 813 | MOUSE: MG-8-50d19 | Transcription |
| SEQ ID No 816 | MOUSE: MG-8-52c9 | Transcription |
| SEQ ID No 818 | MOUSE: MG-8-52i4 | Transcription |
| SEQ ID No 820 | MOUSE: MG-8-53d23 | Transcription |
| SEQ ID No 825 | MOUSE: MG-8-54i8 | Transcription |
| SEQ ID No 826 | MOUSE: MG-8-54o11 | Transcription |
| SEQ ID No 827 | MOUSE: MG-8-54p8 | Transcription |
| SEQ ID No 828 | MOUSE: MG-8-56h3 | Transcription |
| SEQ ID No 838 | MOUSE: MG-8-64i6 | Transcription |
| SEQ ID No 839 | MOUSE: MG-8-68a1 | Transcription |
| SEQ ID No 840 | MOUSE: MG-9-1i19 | Transcription |

SEQ ID-List “Translation”

| | | |
|---|---|---|
| SEQ ID No 6 | MOUSE: MG-11-1k9 | Translation |
| SEQ ID No 13 | MOUSE: MG-12-1a19 | Translation |
| SEQ ID No 15 | MOUSE: MG-12-1e9 | Translation |
| SEQ ID No 22 | MOUSE: MG-13-1m9 | Translation |
| SEQ ID No 36 | MOUSE: MG-14-2n22 | Translation |
| SEQ ID No 45 | MOUSE: MG-14-4e8 | Translation |
| SEQ ID No 50 | MOUSE: MG-14-5f17 | Translation |
| SEQ ID No 74 | MOUSE: MG-16-5i4 | Translation |
| SEQ ID No 75 | MOUSE: MG-16-5j12 | Translation |
| SEQ ID No 76 | MOUSE: MG-16-5n6 | Translation |
| SEQ ID No 81 | MOUSE: MG-16-6d20 | Translation |
| SEQ ID No 83 | MOUSE: MG-16-6i6 | Translation |
| SEQ ID No 89 | MOUSE: MG-16-7h24 | Translation |
| SEQ ID No 90 | MOUSE: MG-16-7h7 | Translation |
| SEQ ID No 91 | MOUSE: MG-16-7j8 | Translation |
| SEQ ID No 93 | MOUSE: MG-16-7o11 | Translation |
| SEQ ID No 97 | MOUSE: MG-16-9b10 | Translation |
| SEQ ID No 129 | MOUSE: MG-3-10h13 | Translation |
| SEQ ID No 131 | MOUSE: MG-3-10k5 | Translation |
| SEQ ID No 133 | MOUSE: MG-3-10p9 | Translation |
| SEQ ID No 153 | MOUSE: MG-3-120g12 | Translation |
| SEQ ID No 156 | MOUSE: MG-3-122e22 | Translation |
| SEQ ID No 166 | MOUSE: MG-3-139d2 | Translation |
| SEQ ID No 171 | MOUSE: MG-3-140g13 | Translation |
| SEQ ID No 175 | MOUSE: MG-3-143d7 | Translation |
| SEQ ID No 178 | MOUSE: MG-3-14c24 | Translation |
| SEQ ID No 179 | MOUSE: MG-3-14d5 | Translation |
| SEQ ID No 192 | MOUSE: MG-3-1j20 | Translation |
| SEQ ID No 196 | MOUSE: MG-3-21i18 | Translation |
| SEQ ID No 201 | MOUSE: MG-3-22k6 | Translation |
| SEQ ID No 204 | MOUSE: MG-3-22n13 | Translation |
| SEQ ID No 207 | MOUSE: MG-3-23e1 | Translation |
| SEQ ID No 208 | MOUSE: MG-3-23j5 | Translation |
| SEQ ID No 210 | MOUSE: MG-3-23p7 | Translation |
| SEQ ID No 219 | MOUSE: MG-3-25f12 | Translation |
| SEQ ID No 224 | MOUSE: MG-3-26d7 | Translation |
| SEQ ID No 226 | MOUSE: MG-3-26l5 | Translation |
| SEQ ID No 227 | MOUSE: MG-3-26n4 | Translation |
| SEQ ID No 236 | MOUSE: MG-3-29a3 | Translation |
| SEQ ID No 247 | MOUSE: MG-3-30h20 | Translation |
| SEQ ID No 249 | MOUSE: MG-3-31c24 | Translation |
| SEQ ID No 251 | MOUSE: MG-3-32d21 | Translation |
| SEQ ID No 269 | MOUSE: MG-3-37o13 | Translation |
| SEQ ID No 279 | MOUSE: MG-3-41b21 | Translation |
| SEQ ID No 309 | MOUSE: MG-3-49g2 | Translation |
| SEQ ID No 311 | MOUSE: MG-3-49k8 | Translation |
| SEQ ID No 312 | MOUSE: MG-3-49o23 | Translation |
| SEQ ID No 316 | MOUSE: MG-3-4g14 | Translation |
| SEQ ID No 317 | MOUSE: MG-3-4g22 | Translation |
| SEQ ID No 319 | MOUSE: MG-3-4i2 | Translation |
| SEQ ID No 321 | MOUSE: MG-3-4m17 | Translation |
| SEQ ID No 332 | MOUSE: MG-3-52l2 | Translation |
| SEQ ID No 334 | MOUSE: MG-3-54f11 | Translation |
| SEQ ID No 340 | MOUSE: MG-3-5b3 | Translation |
| SEQ ID No 341 | MOUSE: MG-3-5b4 | Translation |
| SEQ ID No 345 | MOUSE: MG-3-5g23 | Translation |
| SEQ ID No 346 | MOUSE: MG-3-5k15 | Translation |
| SEQ ID No 347 | MOUSE: MG-3-61i4 | Translation |
| SEQ ID No 352 | MOUSE: MG-3-63k21 | Translation |
| SEQ ID No 357 | MOUSE: MG-3-66j9 | Translation |
| SEQ ID No 366 | MOUSE: MG-3-71g13 | Translation |
| SEQ ID No 371 | MOUSE: MG-3-73n22 | Translation |
| SEQ ID No 373 | MOUSE: MG-3-74a2 | Translation |
| SEQ ID No 377 | MOUSE: MG-3-75a4 | Translation |
| SEQ ID No 383 | MOUSE: MG-3-76b14 | Translation |
| SEQ ID No 390 | MOUSE: MG-3-79b21 | Translation |
| SEQ ID No 392 | MOUSE: MG-3-79j2 | Translation |
| SEQ ID No 396 | MOUSE: MG-3-7g1 | Translation |
| SEQ ID No 397 | MOUSE: MG-3-7l16 | Translation |
| SEQ ID No 406 | MOUSE: MG-3-81o13 | Translation |
| SEQ ID No 414 | MOUSE: MG-3-85e13 | Translation |
| SEQ ID No 418 | MOUSE: MG-3-88c2 | Translation |
| SEQ ID No 433 | MOUSE: MG-3-91j21 | Translation |
| SEQ ID No 434 | MOUSE: MG-3-92p15 | Translation |
| SEQ ID No 436 | MOUSE: MG-3-93m8 | Translation |
| SEQ ID No 442 | MOUSE: MG-3-9f22 | Translation |
| SEQ ID No 446 | MOUSE: MG-3-9o4 | Translation |
| SEQ ID No 448 | MOUSE: MG-4-145j12 | Translation |
| SEQ ID No 451 | MOUSE: MG-4-145o11 | Translation |
| SEQ ID No 456 | MOUSE: MG-4-146n10 | Translation |
| SEQ ID No 464 | MOUSE: MG-4-148n17 | Translation |
| SEQ ID No 467 | MOUSE: MG-4-1i7 | Translation |
| SEQ ID No 468 | MOUSE: MG-4-1k23 | Translation |
| SEQ ID No 481 | MOUSE: MG-4-5e23 | Translation |
| SEQ ID No 485 | MOUSE: MG-4-86c3 | Translation |
| SEQ ID No 512 | MOUSE: MG-6-21f24 | Translation |
| SEQ ID No 515 | MOUSE: MG-6-22n7 | Translation |
| SEQ ID No 518 | MOUSE: MG-6-24o16 | Translation |
| SEQ ID No 523 | MOUSE: MG-6-2j1 | Translation |
| SEQ ID No 525 | MOUSE: MG-6-2l11 | Translation |
| SEQ ID No 533 | MOUSE: MG-6-32e12 | Translation |
| SEQ ID No 536 | MOUSE: MG-6-33d5 | Translation |
| SEQ ID No 551 | MOUSE: MG-6-38p3 | Translation |
| SEQ ID No 554 | MOUSE: MG-6-3c6 | Translation |
| SEQ ID No 562 | MOUSE: MG-6-3o1 | Translation |
| SEQ ID No 564 | MOUSE: MG-6-3o16 | Translation |
| SEQ ID No 570 | MOUSE: MG-6-41g11 | Translation |
| SEQ ID No 574 | MOUSE: MG-6-41o19 | Translation |
| SEQ ID No 582 | MOUSE: MG-6-44a19 | Translation |
| SEQ ID No 584 | MOUSE: MG-6-44i2 | Translation |
| SEQ ID No 601 | MOUSE: MG-6-55g3 | Translation |
| SEQ ID No 610 | MOUSE: MG-6-58p10 | Translation |
| SEQ ID No 620 | MOUSE: MG-6-63c21 | Translation |
| SEQ ID No 621 | MOUSE: MG-6-64d16 | Translation |
| SEQ ID No 623 | MOUSE: MG-6-64p15 | Translation |
| SEQ ID No 650 | MOUSE: MG-6-80j23 | Translation |
| SEQ ID No 657 | MOUSE: MG-6-82l17 | Translation |
| SEQ ID No 661 | MOUSE: MG-6-83n11 | Translation |
| SEQ ID No 667 | MOUSE: MG-6-88k9 | Translation |
| SEQ ID No 674 | MOUSE: MG-6-90g23 | Translation |
| SEQ ID No 680 | MOUSE: MG-6-92a9 | Translation |
| SEQ ID No 681 | MOUSE: MG-8-10b11 | Translation |
| SEQ ID No 683 | MOUSE: MG-8-117a15 | Translation |
| SEQ ID No 685 | MOUSE: MG-8-117g16 | Translation |
| SEQ ID No 686 | MOUSE: MG-8-117i1 | Translation |
| SEQ ID No 687 | MOUSE: MG-8-117k3 | Translation |
| SEQ ID No 690 | MOUSE: MG-8-117m23 | Translation |
| SEQ ID No 691 | MOUSE: MG-8-117m3 | Translation |
| SEQ ID No 694 | MOUSE: MG-8-118j13 | Translation |
| SEQ ID No 700 | MOUSE: MG-8-11l12 | Translation |
| SEQ ID No 713 | MOUSE: MG-8-13m24 | Translation |
| SEQ ID No 716 | MOUSE: MG-8-14o16 | Translation |
| SEQ ID No 729 | MOUSE: MG-8-1e21 | Translation |
| SEQ ID No 730 | MOUSE: MG-8-1e23 | Translation |
| SEQ ID No 732 | MOUSE: MG-8-1i19 | Translation |
| SEQ ID No 735 | MOUSE: MG-8-21e1 | Translation |
| SEQ ID No 738 | MOUSE: MG-8-22p22 | Translation |
| SEQ ID No 754 | MOUSE: MG-8-27p12 | Translation |
| SEQ ID No 756 | MOUSE: MG-8-28g9 | Translation |
| SEQ ID No 758 | MOUSE: MG-8-29d18 | Translation |
| SEQ ID No 766 | MOUSE: MG-8-32a23 | Translation |
| SEQ ID No 769 | MOUSE: MG-8-32g19 | Translation |
| SEQ ID No 772 | MOUSE: MG-8-33c4 | Translation |
| SEQ ID No 777 | MOUSE: MG-8-34b8 | Translation |
| SEQ ID No 779 | MOUSE: MG-8-34p22 | Translation |
| SEQ ID No 789 | MOUSE: MG-8-38o15 | Translation |
| SEQ ID No 795 | MOUSE: MG-8-40e9 | Translation |
| SEQ ID No 803 | MOUSE: MG-8-44p6 | Translation |
| SEQ ID No 805 | MOUSE: MG-8-45n10 | Translation |
| SEQ ID No 807 | MOUSE: MG-8-47d16 | Translation |
| SEQ ID No 814 | MOUSE: MG-8-51a18 | Translation |
| SEQ ID No 824 | MOUSE: MG-8-54i18 | Translation |
| SEQ ID No 832 | MOUSE: MG-8-60f17 | Translation |

SEQ ID-List “Transport-and Binding Proteins”

| | | |
|---|---|---|
| SEQ ID No 1 | MOUSE: MG-11-1a21 | Transport and binding proteins |
| SEQ ID No 2 | MOUSE: MG-11-1c3 | Transport and binding proteins |
| SEQ ID No 3 | MOUSE: MG-11-1e15 | Transport and binding proteins |
| SEQ ID No 4 | MOUSE: MG-11-1e9 | Transport and binding proteins |
| SEQ ID No 5 | MOUSE: MG-11-1g9 | Transport and binding proteins |
| SEQ ID No 8 | MOUSE: MG-11-1o3 | Transport and binding proteins |
| SEQ ID No 9 | MOUSE: MG-11-2d5 | Transport and binding proteins |
| SEQ ID No 10 | MOUSE: MG-11-2h21 | Transport and binding proteins |
| SEQ ID No 11 | MOUSE: MG-11-2n19 | Transport and binding proteins |
| SEQ ID No 12 | MOUSE: MG-11-2p7 | Transport and binding proteins |
| SEQ ID No 14 | MOUSE: MG-12-1c11 | Transport and binding proteins |
| SEQ ID No 16 | MOUSE: MG-12-1g3 | Transport and binding proteins |
| SEQ ID No 17 | MOUSE: MG-12-1g9 | Transport and binding proteins |
| SEQ ID No 18 | MOUSE: MG-12-1i15 | Transport and binding proteins |
| SEQ ID No 19 | MOUSE: MG-12-1k17 | Transport and binding proteins |
| SEQ ID No 20 | MOUSE: MG-13-1c17 | Transport and binding proteins |
| SEQ ID No 23 | MOUSE: MG-13-2a16 | Transport and binding proteins |
| SEQ ID No 24 | MOUSE: MG-13-2f1 | Transport and binding proteins |
| SEQ ID No 28 | MOUSE: MG-13-6j20 | Transport and binding proteins |
| SEQ ID No 30 | MOUSE: MG-13-6p18 | Transport and binding proteins |
| SEQ ID No 31 | MOUSE: MG-13-6p3 | Transport and binding proteins |
| SEQ ID No 32 | MOUSE: MG-14-1k21 | Transport and binding proteins |
| SEQ ID No 33 | MOUSE: MG-14-2c6 | Transport and binding proteins |
| SEQ ID No 34 | MOUSE: MG-14-2k21 | Transport and binding proteins |
| SEQ ID No 35 | MOUSE: MG-14-2n18 | Transport and binding proteins |
| SEQ ID No 37 | MOUSE: MG-14-2o15 | Transport and binding proteins |
| SEQ ID No 38 | MOUSE: MG-14-3b13 | Transport and binding proteins |
| SEQ ID No 39 | MOUSE: MG-14-3b19 | Transport and binding proteins |
| SEQ ID No 40 | MOUSE: MG-14-3j13 | Transport and binding proteins |
| SEQ ID No 41 | MOUSE: MG-14-3j17 | Transport and binding proteins |
| SEQ ID No 42 | MOUSE: MG-14-3k15 | Transport and binding proteins |
| SEQ ID No 43 | MOUSE: MG-14-4b13 | Transport and binding proteins |
| SEQ ID No 44 | MOUSE: MG-14-4c7 | Transport and binding proteins |
| SEQ ID No 47 | MOUSE: MG-14-5a20 | Transport and binding proteins |
| SEQ ID No 48 | MOUSE: MG-14-5d13 | Transport and binding proteins |
| SEQ ID No 49 | MOUSE: MG-14-5f16 | Transport and binding proteins |
| SEQ ID No 51 | MOUSE: MG-14-5h15 | Transport and binding proteins |
| SEQ ID No 55 | MOUSE: MG-15-2f14 | Transport and binding proteins |
| SEQ ID No 56 | MOUSE: MG-15-2j1 | Transport and binding proteins |
| SEQ ID No 57 | MOUSE: MG-15-2p22 | Transport and binding proteins |
| SEQ ID No 58 | MOUSE: MG-15-3a6 | Transport and binding proteins |
| SEQ ID No 59 | MOUSE: MG-15-3f12 | Transport and binding proteins |
| SEQ ID No 60 | MOUSE: MG-15-3k2 | Transport and binding proteins |
| SEQ ID No 61 | MOUSE: MG-15-3n13 | Transport and binding proteins |
| SEQ ID No 66 | MOUSE: MG-16-3f2 | Transport and binding proteins |
| SEQ ID No 67 | MOUSE: MG-16-3h10 | Transport and binding proteins |
| SEQ ID No 68 | MOUSE: MG-16-4a19 | Transport and binding proteins |
| SEQ ID No 70 | MOUSE: MG-16-4e24 | Transport and binding proteins |
| SEQ ID No 72 | MOUSE: MG-16-5g12 | Transport and binding proteins |
| SEQ ID No 77 | MOUSE: MG-16-5o12 | Transport and binding proteins |
| SEQ ID No 78 | MOUSE: MG-16-5p22 | Transport and binding proteins |
| SEQ ID No 80 | MOUSE: MG-16-6b4 | Transport and binding proteins |
| SEQ ID No 82 | MOUSE: MG-16-6f22 | Transport and binding proteins |
| SEQ ID No 84 | MOUSE: MG-16-6o13 | Transport and binding proteins |
| SEQ ID No 86 | MOUSE: MG-16-7d5 | Transport and binding proteins |
| SEQ ID No 87 | MOUSE: MG-16-7d8 | Transport and binding proteins |
| SEQ ID No 88 | MOUSE: MG-16-7g14 | Transport and binding proteins |
| SEQ ID No 95 | MOUSE: MG-16-9a14 | Transport and binding proteins |
| SEQ ID No 96 | MOUSE: MG-16-9a20 | Transport and binding proteins |
| SEQ ID No 99 | MOUSE: MG-16-9e1 | Transport and binding proteins |
| SEQ ID No 101 | MOUSE: MG-17-1a5 | Transport and binding proteins |
| SEQ ID No 102 | MOUSE: MG-19-1a15 | Transport and binding proteins |
| SEQ ID No 103 | MOUSE: MG-19-1a5 | Transport and binding proteins |
| SEQ ID No 105 | MOUSE: MG-19-2e7 | Transport and binding proteins |
| SEQ ID No 106 | MOUSE: MG-19-2i17 | Transport and binding proteins |
| SEQ ID No 107 | MOUSE: MG-19-2m13 | Transport and binding proteins |
| SEQ ID No 108 | MOUSE: MG-20-1i13 | Transport and binding proteins |
| SEQ ID No 109 | MOUSE: MG-20-1k23 | Transport and binding proteins |
| SEQ ID No 110 | MOUSE: MG-20-1k3 | Transport and binding proteins |
| SEQ ID No 111 | MOUSE: MG-20-1m3 | Transport and binding proteins |
| SEQ ID No 113 | MOUSE: MG-3-102c24 | Transport and binding proteins |
| SEQ ID No 114 | MOUSE: MG-3-102g18 | Transport and binding proteins |
| SEQ ID No 117 | MOUSE: MG-3-106b23 | Transport and binding proteins |
| SEQ ID No 120 | MOUSE: MG-3-107o14 | Transport and binding proteins |
| SEQ ID No 124 | MOUSE: MG-3-108l7 | Transport and binding proteins |
| SEQ ID No 128 | MOUSE: MG-3-10e15 | Transport and binding proteins |
| SEQ ID No 132 | MOUSE: MG-3-10m13 | Transport and binding proteins |
| SEQ ID No 135 | MOUSE: MG-3-110k6 | Transport and binding proteins |
| SEQ ID No 136 | MOUSE: MG-3-112g2 | Transport and binding proteins |
| SEQ ID No 137 | MOUSE: MG-3-113c13 | Transport and binding proteins |
| SEQ ID No 141 | MOUSE: MG-3-114h8 | Transport and binding proteins |
| SEQ ID No 143 | MOUSE: MG-3-116a13 | Transport and binding proteins |
| SEQ ID No 145 | MOUSE: MG-3-118i20 | Transport and binding proteins |
| SEQ ID No 146 | MOUSE: MG-3-119l11 | Transport and binding proteins |
| SEQ ID No 147 | MOUSE: MG-3-119n6 | Transport and binding proteins |
| SEQ ID No 148 | MOUSE: MG-3-11c14 | Transport and binding proteins |
| SEQ ID No 149 | MOUSE: MG-3-11h21 | Transport and binding proteins |
| SEQ ID No 150 | MOUSE: MG-3-11i23 | Transport and binding proteins |
| SEQ ID No 152 | MOUSE: MG-3-11m11 | Transport and binding proteins |
| SEQ ID No 154 | MOUSE: MG-3-121b13 | Transport and binding proteins |
| SEQ ID No 157 | MOUSE: MG-3-122j2 | Transport and binding proteins |
| SEQ ID No 158 | MOUSE: MG-3-123g9 | Transport and binding proteins |
| SEQ ID No 159 | MOUSE: MG-3-12a20 | Transport and binding proteins |
| SEQ ID No 167 | MOUSE: MG-3-139o21 | Transport and binding proteins |
| SEQ ID No 173 | MOUSE: MG-3-141p23 | Transport and binding proteins |
| SEQ ID No 177 | MOUSE: MG-3-14b17 | Transport and binding proteins |
| SEQ ID No 181 | MOUSE: MG-3-14h14 | Transport and binding proteins |
| SEQ ID No 184 | MOUSE: MG-3-16d22 | Transport and binding proteins |
| SEQ ID No 188 | MOUSE: MG-3-18o12 | Transport and binding proteins |
| SEQ ID No 191 | MOUSE: MG-3-1h10 | Transport and binding proteins |
| SEQ ID No 193 | MOUSE: MG-3-20a17 | Transport and binding proteins |
| SEQ ID No 195 | MOUSE: MG-3-20o24 | Transport and binding proteins |
| SEQ ID No 198 | MOUSE: MG-3-21o12 | Transport and binding proteins |
| SEQ ID No 200 | MOUSE: MG-3-22j14 | Transport and binding proteins |
| SEQ ID No 205 | MOUSE: MG-3-22o14 | Transport and binding proteins |
| SEQ ID No 211 | MOUSE: MG-3-24h15 | Transport and binding proteins |
| SEQ ID No 214 | MOUSE: MG-3-24o15 | Transport and binding proteins |
| SEQ ID No 222 | MOUSE: MG-3-25p22 | Transport and binding proteins |
| SEQ ID No 230 | MOUSE: MG-3-27i21 | Transport and binding proteins |
| SEQ ID No 231 | MOUSE: MG-3-27m22 | Transport and binding proteins |
| SEQ ID No 232 | MOUSE: MG-3-28a21 | Transport and binding proteins |
| SEQ ID No 234 | MOUSE: MG-3-28g4 | Transport and binding proteins |
| SEQ ID No 237 | MOUSE: MG-3-29e3 | Transport and binding proteins |
| SEQ ID No 239 | MOUSE: MG-3-29m14 | Transport and binding proteins |
| SEQ ID No 243 | MOUSE: MG-3-2j13 | Transport and binding proteins |
| SEQ ID No 245 | MOUSE: MG-3-2m2 | Transport and binding proteins |
| SEQ ID No 246 | MOUSE: MG-3-30c21 | Transport and binding proteins |
| SEQ ID No 248 | MOUSE: MG-3-30m22 | Transport and binding proteins |
| SEQ ID No 253 | MOUSE: MG-3-32h9 | Transport and binding proteins |
| SEQ ID No 256 | MOUSE: MG-3-32p8 | Transport and binding proteins |
| SEQ ID No 258 | MOUSE: MG-3-34e1 | Transport and binding proteins |
| SEQ ID No 259 | MOUSE: MG-3-34p17 | Transport and binding proteins |
| SEQ ID No 260 | MOUSE: MG-3-35b15 | Transport and binding proteins |
| SEQ ID No 261 | MOUSE: MG-3-35p13 | Transport and binding proteins |
| SEQ ID No 263 | MOUSE: MG-3-36h6 | Transport and binding proteins |
| SEQ ID No 265 | MOUSE: MG-3-37j5 | Transport and binding proteins |
| SEQ ID No 271 | MOUSE: MG-3-38m24 | Transport and binding proteins |
| SEQ ID No 273 | MOUSE: MG-3-39a16 | Transport and binding proteins |
| SEQ ID No 284 | MOUSE: MG-3-43i9 | Transport and binding proteins |
| SEQ ID No 286 | MOUSE: MG-3-44b1 | Transport and binding proteins |
| SEQ ID No 287 | MOUSE: MG-3-44f13 | Transport and binding proteins |
| SEQ ID No 289 | MOUSE: MG-3-44l17 | Transport and binding proteins |
| SEQ ID No 292 | MOUSE: MG-3-45n20 | Transport and binding proteins |
| SEQ ID No 293 | MOUSE: MG-3-45o3 | Transport and binding proteins |
| SEQ ID No 298 | MOUSE: MG-3-47c5 | Transport and binding proteins |
| SEQ ID No 299 | MOUSE: MG-3-47e5 | Transport and binding proteins |
| SEQ ID No 301 | MOUSE: MG-3-48a13 | Transport and binding proteins |
| SEQ ID No 302 | MOUSE: MG-3-48k1 | Transport and binding proteins |
| SEQ ID No 306 | MOUSE: MG-3-48o14 | Transport and binding proteins |
| SEQ ID No 314 | MOUSE: MG-3-4c20 | Transport and binding proteins |
| SEQ ID No 318 | MOUSE: MG-3-4i17 | Transport and binding proteins |
| SEQ ID No 320 | MOUSE: MG-3-4l8 | Transport and binding proteins |
| SEQ ID No 323 | MOUSE: MG-3-50k13 | Transport and binding proteins |
| SEQ ID No 327 | MOUSE: MG-3-51b24 | Transport and binding proteins |
| SEQ ID No 328 | MOUSE: MG-3-51g15 | Transport and binding proteins |
| SEQ ID No 330 | MOUSE: MG-3-51l8 | Transport and binding proteins |
| SEQ ID No 335 | MOUSE: MG-3-54h15 | Transport and binding proteins |
| SEQ ID No 337 | MOUSE: MG-3-56j8 | Transport and binding proteins |
| SEQ ID No 338 | MOUSE: MG-3-56n13 | Transport and binding proteins |
| SEQ ID No 343 | MOUSE: MG-3-5e21 | Transport and binding proteins |
| SEQ ID No 344 | MOUSE: MG-3-5g1 | Transport and binding proteins |
| SEQ ID No 348 | MOUSE: MG-3-61p4 | Transport and binding proteins |
| SEQ ID No 350 | MOUSE: MG-3-62j3 | Transport and binding proteins |
| SEQ ID No 353 | MOUSE: MG-3-63p1 | Transport and binding proteins |
| SEQ ID No 356 | MOUSE: MG-3-66i9 | Transport and binding proteins |
| SEQ ID No 360 | MOUSE: MG-3-68i14 | Transport and binding proteins |
| SEQ ID No 361 | MOUSE: MG-3-69a7 | Transport and binding proteins |
| SEQ ID No 362 | MOUSE: MG-3-69n2 | Transport and binding proteins |
| SEQ ID No 363 | MOUSE: MG-3-6b5 | Transport and binding proteins |
| SEQ ID No 364 | MOUSE: MG-3-70g5 | Transport and binding proteins |
| SEQ ID No 365 | MOUSE: MG-3-70n24 | Transport and binding proteins |
| SEQ ID No 369 | MOUSE: MG-3-73b19 | Transport and binding proteins |
| SEQ ID No 370 | MOUSE: MG-3-73f21 | Transport and binding proteins |
| SEQ ID No 374 | MOUSE: MG-3-74d18 | Transport and binding proteins |
| SEQ ID No 375 | MOUSE: MG-3-74f18 | Transport and binding proteins |
| SEQ ID No 385 | MOUSE: MG-3-76f2 | Transport and binding proteins |
| SEQ ID No 386 | MOUSE: MG-3-76n17 | Transport and binding proteins |
| SEQ ID No 388 | MOUSE: MG-3-77l18 | Transport and binding proteins |
| SEQ ID No 391 | MOUSE: MG-3-79e7 | Transport and binding proteins |
| SEQ ID No 394 | MOUSE: MG-3-79n2 | Transport and binding proteins |
| SEQ ID No 404 | MOUSE: MG-3-81g17 | Transport and binding proteins |
| SEQ ID No 407 | MOUSE: MG-3-82c19 | Transport and binding proteins |
| SEQ ID No 408 | MOUSE: MG-3-82l18 | Transport and binding proteins |
| SEQ ID No 409 | MOUSE: MG-3-82l20 | Transport and binding proteins |
| SEQ ID No 413 | MOUSE: MG-3-85c3 | Transport and binding proteins |
| SEQ ID No 416 | MOUSE: MG-3-86a7 | Transport and binding proteins |
| SEQ ID No 423 | MOUSE: MG-3-89m14 | Transport and binding proteins |
| SEQ ID No 424 | MOUSE: MG-3-8f7 | Transport and binding proteins |
| SEQ ID No 427 | MOUSE: MG-3-8l23 | Transport and binding proteins |
| SEQ ID No 428 | MOUSE: MG-3-90d1 | Transport and binding proteins |
| SEQ ID No 431 | MOUSE: MG-3-91 a10 | Transport and binding proteins |
| SEQ ID No 440 | MOUSE: MG-3-96e8 | Transport and binding proteins |
| SEQ ID No 443 | MOUSE: MG-3-9g4 | Transport and binding proteins |
| SEQ ID No 445 | MOUSE: MG-3-9h2 | Transport and binding proteins |
| SEQ ID No 447 | MOUSE: MG-4-145f3 | Transport and binding proteins |
| SEQ ID No 453 | MOUSE: MG-4-146f15 | Transport and binding proteins |
| SEQ ID No 455 | MOUSE: MG-4-146l4 | Transport and binding proteins |
| SEQ ID No 458 | MOUSE: MG-4-147d22 | Transport and binding proteins |
| SEQ ID No 459 | MOUSE: MG-4-148b6 | Transport and binding proteins |
| SEQ ID No 460 | MOUSE: MG-4-148b7 | Transport and binding proteins |
| SEQ ID No 463 | MOUSE: MG-4-148k6 | Transport and binding proteins |
| SEQ ID No 466 | MOUSE: MG-4-149e2 | Transport and binding proteins |
| SEQ ID No 469 | MOUSE: MG-4-2j8 | Transport and binding proteins |
| SEQ ID No 470 | MOUSE: MG-4-2o11 | Transport and binding proteins |
| SEQ ID No 472 | MOUSE: MG-4-3d20 | Transport and binding proteins |
| SEQ ID No 473 | MOUSE: MG-4-3f17 | Transport and binding proteins |
| SEQ ID No 474 | MOUSE: MG-4-3l13 | Transport and binding proteins |
| SEQ ID No 475 | MOUSE: MG-4-4g14 | Transport and binding proteins |
| SEQ ID No 476 | MOUSE: MG-4-4h1 | Transport and binding proteins |
| SEQ ID No 478 | MOUSE: MG-4-4i10 | Transport and binding proteins |
| SEQ ID No 480 | MOUSE: MG-4-5c24 | Transport and binding proteins |
| SEQ ID No 482 | MOUSE: MG-4-5i13 | Transport and binding proteins |
| SEQ ID No 484 | MOUSE: MG-4-6d16 | Transport and binding proteins |
| SEQ ID No 486 | MOUSE: MG-6-10g10 | Transport and binding proteins |
| SEQ ID No 489 | MOUSE: MG-6-11a6 | Transport and binding proteins |
| SEQ ID No 490 | MOUSE: MG-6-12c20 | Transport and binding proteins |
| SEQ ID No 491 | MOUSE: MG-6-13g8 | Transport and binding proteins |
| SEQ ID No 493 | MOUSE: MG-6-13k5 | Transport and binding proteins |
| SEQ ID No 494 | MOUSE: MG-6-14h9 | Transport and binding proteins |
| SEQ ID No 495 | MOUSE: MG-6-14k2 | Transport and binding proteins |
| SEQ ID No 497 | MOUSE: MG-6-15f10 | Transport and binding proteins |
| SEQ ID No 498 | MOUSE: MG-6-15m21 | Transport and binding proteins |
| SEQ ID No 499 | MOUSE: MG-6-15n12 | Transport and binding proteins |
| SEQ ID No 500 | MOUSE: MG-6-16c3 | Transport and binding proteins |
| SEQ ID No 501 | MOUSE: MG-6-16i3 | Transport and binding proteins |
| SEQ ID No 505 | MOUSE: MG-6-18c1 | Transport and binding proteins |
| SEQ ID No 507 | MOUSE: MG-6-19l15 | Transport and binding proteins |
| SEQ ID No 508 | MOUSE: MG-6-1g5 | Transport and binding proteins |
| SEQ ID No 510 | MOUSE: MG-6-1m13 | Transport and binding proteins |
| SEQ ID No 511 | MOUSE: MG-6-21e9 | Transport and binding proteins |
| SEQ ID No 513 | MOUSE: MG-6-22g14 | Transport and binding proteins |
| SEQ ID No 516 | MOUSE: MG-6-23i7 | Transport and binding proteins |
| SEQ ID No 517 | MOUSE: MG-6-24b11 | Transport and binding proteins |
| SEQ ID No 520 | MOUSE: MG-6-25i13 | Transport and binding proteins |
| SEQ ID No 522 | MOUSE: MG-6-2g18 | Transport and binding proteins |
| SEQ ID No 528 | MOUSE: MG-6-31b6 | Transport and binding proteins |
| SEQ ID No 529 | MOUSE: MG-6-31c20 | Transport and binding proteins |
| SEQ ID No 532 | MOUSE: MG-6-32c7 | Transport and binding proteins |
| SEQ ID No 543 | MOUSE: MG-6-36e10 | Transport and binding proteins |
| SEQ ID No 544 | MOUSE: MG-6-36h20 | Transport and binding proteins |
| SEQ ID No 546 | MOUSE: MG-6-36m12 | Transport and binding proteins |
| SEQ ID No 548 | MOUSE: MG-6-37m17 | Transport and binding proteins |
| SEQ ID No 549 | MOUSE: MG-6-38n21 | Transport and binding proteins |
| SEQ ID No 550 | MOUSE: MG-6-38n22 | Transport and binding proteins |
| SEQ ID No 552 | MOUSE: MG-6-39b7 | Transport and binding proteins |
| SEQ ID No 553 | MOUSE: MG-6-39o12 | Transport and binding proteins |
| SEQ ID No 555 | MOUSE: MG-6-3f17 | Transport and binding proteins |
| SEQ ID No 556 | MOUSE: MG-6-3h9 | Transport and binding proteins |
| SEQ ID No 558 | MOUSE: MG-6-3j21 | Transport and binding proteins |
| SEQ ID No 559 | MOUSE: MG-6-3k23 | Transport and binding proteins |
| SEQ ID No 560 | MOUSE: MG-6-3m16 | Transport and binding proteins |
| SEQ ID No 561 | MOUSE: MG-6-3n13 | Transport and binding proteins |
| SEQ ID No 563 | MOUSE: MG-6-3o12 | Transport and binding proteins |
| SEQ ID No 567 | MOUSE: MG-6-40h20 | Transport and binding proteins |
| SEQ ID No 568 | MOUSE: MG-6-40n24 | Transport and binding proteins |
| SEQ ID No 569 | MOUSE: MG-6-40p6 | Transport and binding proteins |
| SEQ ID No 577 | MOUSE: MG-6-42e21 | Transport and binding proteins |
| SEQ ID No 579 | MOUSE: MG-6-42j14 | Transport and binding proteins |
| SEQ ID No 580 | MOUSE: MG-6-42k8 | Transport and binding proteins |
| SEQ ID No 585 | MOUSE: MG-6-44k20 | Transport and binding proteins |
| SEQ ID No 586 | MOUSE: MG-6-45k11 | Transport and binding proteins |
| SEQ ID No 589 | MOUSE: MG-6-47k1 | Transport and binding proteins |
| SEQ ID No 591 | MOUSE: MG-6-48f16 | Transport and binding proteins |
| SEQ ID No 592 | MOUSE: MG-6-48n17 | Transport and binding proteins |
| SEQ ID No 593 | MOUSE: MG-6-48o4 | Transport and binding proteins |
| SEQ ID No 595 | MOUSE: MG-6-49c8 | Transport and binding proteins |
| SEQ ID No 596 | MOUSE: MG-6-49m7 | Transport and binding proteins |
| SEQ ID No 597 | MOUSE: MG-6-52n4 | Transport and binding proteins |
| SEQ ID No 598 | MOUSE: MG-6-53m5 | Transport and binding proteins |
| SEQ ID No 599 | MOUSE: MG-6-54d6 | Transport and binding proteins |
| SEQ ID No 600 | MOUSE: MG-6-55f18 | Transport and binding proteins |
| SEQ ID No 602 | MOUSE: MG-6-55j15 | Transport and binding proteins |
| SEQ ID No 603 | MOUSE: MG-6-55o21 | Transport and binding proteins |
| SEQ ID No 605 | MOUSE: MG-6-56e12 | Transport and binding proteins |
| SEQ ID No 606 | MOUSE: MG-6-57d20 | Transport and binding proteins |
| SEQ ID No 607 | MOUSE: MG-6-57g11 | Transport and binding proteins |
| SEQ ID No 608 | MOUSE: MG-6-57i2 | Transport and binding proteins |
| SEQ ID No 611 | MOUSE: MG-6-59c13 | Transport and binding proteins |
| SEQ ID No 613 | MOUSE: MG-6-61c23 | Transport and binding proteins |
| SEQ ID No 615 | MOUSE: MG-6-61h24 | Transport and binding proteins |
| SEQ ID No 617 | MOUSE: MG-6-61l13 | Transport and binding proteins |
| SEQ ID No 618 | MOUSE: MG-6-62e6 | Transport and binding proteins |
| SEQ ID No 619 | MOUSE: MG-6-62k22 | Transport and binding proteins |
| SEQ ID No 624 | MOUSE: MG-6-64p18 | Transport and binding proteins |
| SEQ ID No 625 | MOUSE: MG-6-65n22 | Transport and binding proteins |
| SEQ ID No 626 | MOUSE: MG-6-66l22 | Transport and binding proteins |
| SEQ ID No 627 | MOUSE: MG-6-68i24 | Transport and binding proteins |
| SEQ ID No 628 | MOUSE: MG-6-69f19 | Transport and binding proteins |
| SEQ ID No 629 | MOUSE: MG-6-69k15 | Transport and binding proteins |
| SEQ ID No 630 | MOUSE: MG-6-6n15 | Transport and binding proteins |
| SEQ ID No 632 | MOUSE: MG-6-70i21 | Transport and binding proteins |
| SEQ ID No 633 | MOUSE: MG-6-71g7 | Transport and binding proteins |
| SEQ ID No 636 | MOUSE: MG-6-71o8 | Transport and binding proteins |
| SEQ ID No 637 | MOUSE: MG-6-74c18 | Transport and binding proteins |
| SEQ ID No 639 | MOUSE: MG-6-75a14 | Transport and binding proteins |
| SEQ ID No 641 | MOUSE: MG-6-75c23 | Transport and binding proteins |
| SEQ ID No 644 | MOUSE: MG-6-76d14 | Transport and binding proteins |
| SEQ ID No 645 | MOUSE: MG-6-76f17 | Transport and binding proteins |
| SEQ ID No 646 | MOUSE: MG-6-77b14 | Transport and binding proteins |
| SEQ ID No 648 | MOUSE: MG-6-79i6 | Transport and binding proteins |
| SEQ ID No 649 | MOUSE: MG-6-79m7 | Transport and binding proteins |
| SEQ ID No 651 | MOUSE: MG-6-80k3 | Transport and binding proteins |
| SEQ ID No 652 | MOUSE: MG-6-81g14 | Transport and binding proteins |
| SEQ ID No 653 | MOUSE: MG-6-82c5 | Transport and binding proteins |
| SEQ ID No 654 | MOUSE: MG-6-82f1 | Transport and binding proteins |
| SEQ ID No 655 | MOUSE: MG-6-82h17 | Transport and binding proteins |
| SEQ ID No 656 | MOUSE: MG-6-82k5 | Transport and binding proteins |
| SEQ ID No 658 | MOUSE: MG-6-82m18 | Transport and binding proteins |
| SEQ ID No 660 | MOUSE: MG-6-83m8 | Transport and binding proteins |
| SEQ ID No 662 | MOUSE: MG-6-85j21 | Transport and binding proteins |
| SEQ ID No 664 | MOUSE: MG-6-86o11 | Transport and binding proteins |
| SEQ ID No 665 | MOUSE: MG-6-86p1 | Transport and binding proteins |
| SEQ ID No 666 | MOUSE: MG-6-86p2 | Transport and binding proteins |
| SEQ ID No 668 | MOUSE: MG-6-89a23 | Transport and binding proteins |
| SEQ ID No 672 | MOUSE: MG-6-90d16 | Transport and binding proteins |
| SEQ ID No 675 | MOUSE: MG-6-90m19 | Transport and binding proteins |
| SEQ ID No 676 | MOUSE: MG-6-91d19 | Transport and binding proteins |
| SEQ ID No 677 | MOUSE: MG-6-91l17 | Transport and binding proteins |
| SEQ ID No 678 | MOUSE: MG-6-91l19 | Transport and binding proteins |
| SEQ ID No 682 | MOUSE: MG-8-10l23 | Transport and binding proteins |
| SEQ ID No 684 | MOUSE: MG-8-117c22 | Transport and binding proteins |
| SEQ ID No 688 | MOUSE: MG-8-117l2 | Transport and binding proteins |
| SEQ ID No 693 | MOUSE: MG-8-118h19 | Transport and binding proteins |
| SEQ ID No 695 | MOUSE: MG-8-118k19 | Transport and binding proteins |
| SEQ ID No 696 | MOUSE: MG-8-11 b5 | Transport and binding proteins |
| SEQ ID No 697 | MOUSE: MG-8-11g1 | Transport and binding proteins |
| SEQ ID No 698 | MOUSE: MG-8-11g16 | Transport and binding proteins |
| SEQ ID No 699 | MOUSE: MG-8-11j13 | Transport and binding proteins |
| SEQ ID No 701 | MOUSE: MG-8-11n18 | Transport and binding proteins |
| SEQ ID No 702 | MOUSE: MG-8-12b4 | Transport and binding proteins |
| SEQ ID No 703 | MOUSE: MG-8-12i15 | Transport and binding proteins |
| SEQ ID No 704 | MOUSE: MG-8-12l22 | Transport and binding proteins |
| SEQ ID No 706 | MOUSE: MG-8-12n2 | Transport and binding proteins |
| SEQ ID No 708 | MOUSE: MG-8-12o8 | Transport and binding proteins |
| SEQ ID No 709 | MOUSE: MG-8-13a6 | Transport and binding proteins |
| SEQ ID No 711 | MOUSE: MG-8-13h22 | Transport and binding proteins |
| SEQ ID No 712 | MOUSE: MG-8-13i2 | Transport and binding proteins |
| SEQ ID No 717 | MOUSE: MG-8-15l22 | Transport and binding proteins |
| SEQ ID No 720 | MOUSE: MG-8-16f7 | Transport and binding proteins |
| SEQ ID No 722 | MOUSE: MG-8-17a6 | Transport and binding proteins |
| SEQ ID No 724 | MOUSE: MG-8-19a13 | Transport and binding proteins |
| SEQ ID No 725 | MOUSE: MG-8-19b7 | Transport and binding proteins |
| SEQ ID No 726 | MOUSE: MG-8-1a17 | Transport and binding proteins |
| SEQ ID No 727 | MOUSE: MG-8-1c19 | Transport and binding proteins |
| SEQ ID No 733 | MOUSE: MG-8-20h5 | Transport and binding proteins |
| SEQ ID No 737 | MOUSE: MG-8-22g2 | Transport and binding proteins |
| SEQ ID No 739 | MOUSE: MG-8-23a13 | Transport and binding proteins |
| SEQ ID No 742 | MOUSE: MG-8-23j1 | Transport and binding proteins |
| SEQ ID No 745 | MOUSE: MG-8-24h1 | Transport and binding proteins |
| SEQ ID No 746 | MOUSE: MG-8-25c24 | Transport and binding proteins |
| SEQ ID No 747 | MOUSE: MG-8-25c3 | Transport and binding proteins |
| SEQ ID No 748 | MOUSE: MG-8-25n7 | Transport and binding proteins |
| SEQ ID No 749 | MOUSE: MG-8-26g8 | Transport and binding proteins |
| SEQ ID No 750 | MOUSE: MG-8-26i19 | Transport and binding proteins |
| SEQ ID No 752 | MOUSE: MG-8-27j20 | Transport and binding proteins |
| SEQ ID No 755 | MOUSE: MG-8-28e17 | Transport and binding proteins |
| SEQ ID No 757 | MOUSE: MG-8-29d12 | Transport and binding proteins |
| SEQ ID No 759 | MOUSE: MG-8-29g17 | Transport and binding proteins |
| SEQ ID No 760 | MOUSE: MG-8-30c6 | Transport and binding proteins |
| SEQ ID No 761 | MOUSE: MG-8-31c6 | Transport and binding proteins |
| SEQ ID No 762 | MOUSE: MG-8-31e14 | Transport and binding proteins |
| SEQ ID No 763 | MOUSE: MG-8-31j16 | Transport and binding proteins |
| SEQ ID No 764 | MOUSE: MG-8-31o19 | Transport and binding proteins |
| SEQ ID No 765 | MOUSE: MG-8-31p21 | Transport and binding proteins |
| SEQ ID No 767 | MOUSE: MG-8-32c21 | Transport and binding proteins |
| SEQ ID No 770 | MOUSE: MG-8-32m4 | Transport and binding proteins |
| SEQ ID No 773 | MOUSE: MG-8-33e2 | Transport and binding proteins |
| SEQ ID No 774 | MOUSE: MG-8-33e20 | Transport and binding proteins |
| SEQ ID No 776 | MOUSE: MG-8-34a20 | Transport and binding proteins |
| SEQ ID No 780 | MOUSE: MG-8-35g12 | Transport and binding proteins |
| SEQ ID No 781 | MOUSE: MG-8-35n10 | Transport and binding proteins |
| SEQ ID No 782 | MOUSE: MG-8-36b7 | Transport and binding proteins |
| SEQ ID No 783 | MOUSE: MG-8-36g14 | Transport and binding proteins |
| SEQ ID No 784 | MOUSE: MG-8-36j7 | Transport and binding proteins |
| SEQ ID No 786 | MOUSE: MG-8-36n12 | Transport and binding proteins |
| SEQ ID No 787 | MOUSE: MG-8-38f12 | Transport and binding proteins |
| SEQ ID No 791 | MOUSE: MG-8-39h13 | Transport and binding proteins |
| SEQ ID No 792 | MOUSE: MG-8-39l1 | Transport and binding proteins |
| SEQ ID No 794 | MOUSE: MG-8-40b24 | Transport and binding proteins |
| SEQ ID No 797 | MOUSE: MG-8-40j12 | Transport and binding proteins |
| SEQ ID No 798 | MOUSE: MG-8-41d14 | Transport and binding proteins |
| SEQ ID No 799 | MOUSE: MG-8-42j10 | Transport and binding proteins |
| SEQ ID No 802 | MOUSE: MG-8-44p12 | Transport and binding proteins |
| SEQ ID No 806 | MOUSE: MG-8-46i18 | Transport and binding proteins |
| SEQ ID No 808 | MOUSE: MG-8-47m14 | Transport and binding proteins |
| SEQ ID No 811 | MOUSE: MG-8-49h21 | Transport and binding proteins |
| SEQ ID No 812 | MOUSE: MG-8-49l16 | Transport and binding proteins |
| SEQ ID No 819 | MOUSE: MG-8-52p7 | Transport and binding proteins |
| SEQ ID No 821 | MOUSE: MG-8-53g14 | Transport and binding proteins |
| SEQ ID No 822 | MOUSE: MG-8-53n12 | Transport and binding proteins |
| SEQ ID No 829 | MOUSE: MG-8-59b18 | Transport and binding proteins |
| SEQ ID No 830 | MOUSE: MG-8-59i18 | Transport and binding proteins |
| SEQ ID No 831 | MOUSE: MG-8-5h9 | Transport and binding proteins |
| SEQ ID No 833 | MOUSE: MG-8-60i3 | Transport and binding proteins |
| SEQ ID No 834 | MOUSE: MG-8-61n6 | Transport and binding proteins |
| SEQ ID No 835 | MOUSE: MG-8-62f20 | Transport and binding proteins |
| SEQ ID No 836 | MOUSE: MG-8-63o3 | Transport and binding proteins |
| SEQ ID No 837 | MOUSE: MG-8-63p15 | Transport and binding proteins |

1. Method for producing a gene library, whereat said method comprises the following steps:
(a) Producing a double-stranded cDNA from a mRNA-population, wherein for the synthesis of the first cDNA-strand a primer is used, which at its 5′-end carries a first binding partner of a binding pair with this first binding partner having a binding affinity to a second binding partner of the binding pair, which at its 3′-end provides a poly(dT)-sequence being complementary to the poly(A)-tail of the mRNA, and—between the 5′-end being coupled to the first binding partner and the poly(dT)-sequence—a sequence, which as a double strand comprises a recognition site for a type II restriction enzyme;
(b) Fragmentation of the cDNA obtained in step (a);
(c) Binding the obtained fragments to the second binding partner, which is coupled to a solid carrier;
(d) Ligating a double-stranded adaptor molecule to the fragment end opposing the end with the first binding partner, whereat the adaptor molecule contains a restriction site for a second restriction enzyme;
(e) Performing an in vitro-amplification of the fragments bound to the carrier by means of a primer pair, whereat the first primer of the primer pair is mainly complementary to a strand of the adaptor molecule, and whereat the second primer of the primer pair at its 5′-end provides a first binding partner with an affinity to a second partner of a binding pair and has a sequence largely corresponding to the sequence of the primer of step (a) or a sequence complementary thereto; and
(f) Ligating the obtained products into a vector.

2. Method according to claim 1, wherein—after step (b)—the fragments having the desired length are isolated.

3. Method according to claim 1 or 2, wherein—after step (b)— the obtained (isolated) fragments are provided with blunt ends.

4. Method according to any of the claims 1 to 3, wherein the amplification products of step (e) are cleaved with a type II restriction enzyme, which recognizes the restriction site introduced in step (a).

5. Method according to claim 4, wherein thereafter an adaptor molecule is ligated to the 5′-end of the PCR-products, which was obtained by the cleavage with the type II restriction enzyme.

6. Method according to claim 5, wherein the products are cleaved with a second restriction enzyme, which recognizes the cleavage site introduced in step (d).

7. Method according to claim 1, wherein the binding pair is biotin/avidin or biotin/streptavidin.

8. Method according to claim 1, wherein the type II restriction enzyme is selected from the group consisting of BpmI, AlwI, BpsI, BpvI, Bci VI, BsaI, Bse RI, BsgI, Bsm AI, Earl, EciI, FokI, HgaI, HphI, MboII, PleI, SapI and SfaNI.

9. Method according to any of the claims 1 to 8, wherein the primer for the first strand synthesis is a mixture of primers, which—at the 3′-side of the poly(dT)-sequence—provides the sequence 5′-VN-3′, wherein V is A, C or G and N is A, C, G or T.

10. Method according to any of the claims 1 to 3, wherein the fragmentation in step (b) is a random fragmentation or an enzymatic fragmentation.

11. Method according to claim 10, wherein the fragmentation is accomplished by sonication or an enzymatic (partial) digest.

12. Method according to any of the claims 1 to 11, wherein the length of the fragments in step (b) is within a range of 200 to 600 bp.

13. Method according to any of the claims 1 to 12, wherein the solid carrier in step (c) consists of paramagnetic pearls.

14. Method according to any of the claims 1 to 13, wherein the restriction enzyme in step (d) is a restriction enzyme, which produces overhanging ends and/or which is different from the type II restriction enzyme of step (a).

15. Method according to any of the claims 1 to 14, wherein the in vitro-amplification in step (e) is a PCR.

16. Method according to claim 4 and 5, wherein the cleavage is such accomplished, that the obtained fragment provides an overhanging end with at least 2 adenine residues, whereat the adaptor molecule according to claim 5 provides an overhanging end complementary thereto.

17. Gene library, which can be obtained by a method according to any of the claims 1 to 16.

18. Gene library according to claim 17, wherein at least one sequence of a gene or a part of a gene is present, which codes for a protein being involved in one of the following processes selected from the group of: amino acid synthesis, cellular metabolism, energy metabolism, fatty acid- and phospholipid-metabolism, purine-, pyrimidine-, nucleoside- and nucleotide-synthesis and—degradation, DNA-replication, transcription, translation, protein transport or protein binding,
characterized in that the gene library comprises at least 50 sequences, wherein at least 95% of the sequences of the presents genes or of their parts have a length between 200 and 600 base pairs.

19. Gene library according to claim 17 or 18, wherein sequences of at least 200 genes or of parts thereof are present, the products of which are involved in the same or in various of the processes defined in claim 1.

20. Gene library according to any of the claims 17 to 19, wherein sequences of at least 500 genes or of parts thereof are present, the products of which are involved in the same or in various of the processes defined in claim 1.

21. Gene library according to any of the claims 17 to 20, wherein sequences of at least 800 genes or of parts thereof are present, the products of which are involved in the same or in various of the processes defined in claim 1.

22. Gene library according to any of the claims 17 to 21, wherein the genes or the parts thereof are derived from the mouse, rat, dog, human, pig, hamster or cow.

23. Gene library according to any of the claims 17 to 22, wherein at least 60% of the sequences comprise genes or parts of genes, which are derived from the 3′-region of the mRNA.

24. Gene library according to any of the claims 17 to 23, wherein at least 60% of the genes or of the parts thereof comprise sequences without a poly(A)-tail.

25. Gene library according to any of the claims 17 to 24, wherein the sequences of the genes or of the parts thereof are present in a prokaryotic plasmid.

26. Gene library according to any of the claims 17 to 25, wherein the sequences are double-stranded.

27. Gene library according to any of the claims 17 to 26, wherein sequences of at least 50 genes or of parts thereof are present, which are selected from:
(a) the sequences of the SEQ ID-list “Replication”,
(b) the sequences of the SEQ ID-list “Transcription”,
(c) the sequences of the SEQ ID-list “Translation”, and
(d) the sequences of the SEQ ID-list “Transport- and binding proteins”.

28. Gene library according to claim 27, wherein sequences of at least 200 genes or of parts thereof are present, which are selected from:
(a) the sequences of the SEQ ID-list “Replication”,
(b) the sequences of the SEQ ID-list “Transcription”,
(c) the sequences of the SEQ ID-list “Translation”, and
(d) the sequences of the SEQ ID-list “Transport- and binding proteins”.

29. Gene library according to claim 28, wherein sequences of at least 500 genes or of parts thereof are present, which are selected from:
(a) the sequences of the SEQ ID-list “Replication”,
(b) the sequences of the SEQ ID-list “Transcription”,
(c) the sequences of the SEQ ID-list “Translation”, and
(d) the sequences of the SEQ ID-list “Transport—and binding proteins”.

30. Gene library according to any of the claims 27 to 29, wherein the sequences are selected from genes of at least two of the groups (a) to (d).

31. Gene library according to any of the claims 17 to 30, wherein sequences of at least 50 genes or of parts thereof are present, which are selected from the SEQ ID No. 1 to SEQ ID No. 840.

32. Gene library according to claim 31, wherein sequences of at least 200 genes or of parts thereof are present, which are selected from the SEQ ID No. 1 to SEQ ID No. 840.

33. Gene library according to claim 31 or 32, wherein sequences of at least 500 genes or of parts thereof are present, which are selected from the SEQ ID No. 1 to SEQ ID No. 840.

34. Transformant, which contains a gene library according to any of the claims 17 to 33.
